(19) United States
(12) Patent Application Publication
(10) Pub. No.: US 2021/0291190 A1
(43) Pub. Date: Sep. 23, 2021

(54) THERMOCYCLER REACTION CONTROL

(71) Applicant: DeepDiveBio, Inc., San Diego, CA (US)

(72) Inventors: Tei NEWMAN-LEHMAN, San Diego, CA (US); Alex BATES, San Diego, CA (US); Donald NOVKOV, Encinitas, CA (US); Joshua P. SMITH, San Diego, CA (US); Thomas M. LUGO, San Diego, CA (US)

(21) Appl. No.: 17/259,893

(22) PCT Filed: Jul. 11, 2019
(86) PCT No.: PCT/US2019/041485

§ 371 Date: Jan. 12, 2021

Related U.S. Application Data
(60) Provisional application No. 62/858,948, filed on Jun. 7, 2019.
(60) Provisional application No. 62/697,902, filed on Jul. 13, 2018.

(51) Int. Cl. B01L 7/00; G16B 25/00; C12P 19/34; G06F 16/23

(52) U.S. Cl. CPC B01L 7/525; G16B 25/00; C12P 19/34; G06F 16/23; B01L 2200/148; B01L 2300/0829; B01L 2200/147; B01L 2300/18; B01L 2300/1805; B01L 2300/1894; B01L 2300/023; B01L 2300/0663

(57) ABSTRACT

Provided herein are devices, methods, and systems for polynucleotide synthesis comprising a thermocycler comprising a plurality of individual chambers having a capability to control its own temperature setting and a machine learning for generating a recommendation of a design of experiment for polynucleotide synthesis.

### CROSS REFERENCE TO RELATED APPLICATIONS

The present application claims priority to U.S. Provisional Application Ser. No. 62/697,902, filed Jul. 13, 2018, and U.S. Provisional Application Ser. No. 62/858,948, filed Jun. 7, 2019, the contents of which are hereby incorporated by reference in their entirety.

### BACKGROUND

Custom gene synthesis, or polynucleotide synthesis, provides a powerful tool for research in biology and medicine and for various biotechnology applications. Gene synthesis typically involves building specially designed oligonucleotides, preparing the oligonucleotides and various reagents in a mixture, and assembling the oligonucleotides using a thermocycler. There remains a need for enhancing the quality of the synthesized polynucleotide and reducing the time required to generate the polynucleotides.

### SUMMARY

The devices, methods, and systems provided herein address this need, and provide additional advantages as well. The devices, methods, and systems provided herein improve throughput, reduce cost, and provide simple user interface to facilitate collaboration in polynucleotide synthesis. The devices, methods, and systems may comprise a thermocycler comprising a plurality of individual chambers, where the temperature setting of an individual chamber are controlled independently from the temperature setting of another individual chamber, and a system comprising a machine learning for a polynucleotide synthesis for generating a recommendation of a design of experiment for a polynucleotide synthesis. The devices, methods, and systems provided herein can control parameters of individual wells and is small, modular, and easy to maintain. The devices, methods, and systems provided herein may be compatible with laboratory automation, mobile devices, and laboratory information management software (LIMS) to improve throughput, synthesis quality, and reproducibility. Such devices, methods, and systems may improve quality of the polynucleotide product and success of the polynucleotide synthesis by automation and traceability of steps to reduce human errors and by cloud connectivity for distribution of optimized protocols for polynucleotide synthesis. The devices, methods, and systems provided herein may improve the throughput by running multiple protocols with different steps in parallel and using modular architecture that makes it easy to scale. The devices, methods, and systems provided herein may reduce costs by using fewer instruments where each well serves as its own device and has an independent functionality from another well. The devices, methods, and systems provided herein may also reduce costs by reducing labor where the device can fit on a robot deck and is compatible with automated liquid handlers.

Provided herein are devices, system, and methods for thermocycling for polynucleotide synthesis, comprising a plurality of reaction chambers, wherein a temperature setting of an individual reaction chamber can be controlled independent from a temperature setting of another individual reaction chamber. In some embodiments, the device comprises a rotating baseplate, a motor, and a rotating actuator, wherein the plurality of reaction chambers is configured to rotate using the rotating baseplate, the motor, and the rotating actuator. In some embodiments, the device comprises a microprocessor, thermoelectric modules, thermistors (temperature sensors), a heated lid, a heat sink fan, and a power coupling assembly to provide control of the temperature setting of the individual reaction chambers. In some embodiments, the thermoelectric module comprises a thermoelectric cooler (TEC) board and a heat sink, wherein the TEC board comprises a plurality of thermal adhesive pads on a backing material. In some embodiments, the plurality of thermal adhesive pads are spaced apart in similarly as the plurality of reaction chambers. In some embodiments, the thermistor is in contact with an outer wall of the individual reaction chamber. In some embodiments, the device comprises a touchscreen display, a camera, or a microphone, or a combination thereof for interfacing with a user. In some embodiments, the device comprises an online database comprising an artificial intelligence, wherein the online database stores data from a plurality of polynucleotide synthesis reactions, wherein the data comprises reagent conditions, reaction chamber conditions, and quality scores of polynucleotide products. In some embodiments, the online database connects to a web-based application, wherein the web-based application takes an input from a user and displays an output to a user. In some embodiments, the device comprises 96 individual reaction chambers. In some embodiments, the temperature setting of the individual reaction chambers is controllable to at least 0.5° C. In some embodiments, the temperature setting of the individual reaction chambers is controllable to about 0.05° C. In some embodiments, the temperature setting of the individual reaction chambers has a temperature ramp at least 1° C./s. In some embodiments, the temperature setting of the individual reaction chambers has a temperature ramp of about 5° C./s. In some embodiments, the device interfaces with an online database comprising an artificial intelligence, wherein the artificial intelligence generates a report of a recommendation to synthesize a polynucleotide of interest having a high fidelity and wherein the recommendation comprises reagent conditions and reaction chamber conditions. In some embodiments, the generating of the report of the recommendation by a computing system comprises: determining, by artificial intelligence, reagents and reaction chamber conditions to include in the recommendation based on the data of reagents and the data of reaction chamber conditions in the artificial intelligence; and determining, by artificial intelligence, a sequence of the connections of the data of reagents and the data of reaction chamber conditions that provides a polynucleotide quality score above a threshold score as the recommendation. In some embodiments, the device interfaces with the online database by a touchscreen display, a camera, or a microphone, or a combination thereof. In some embodiments, the touchscreen display, the camera, or the microphone, or a combination thereof transmits an input from a user to the online database and displays the report to the user. In some embodiments, the user can modify the report using the touchscreen display, the camera, or the microphone, or a combination thereof before the report is executed by the device. In some embodiments, the online database connects to a web-based application, wherein the web-based application takes an input from a user and displays an output to a user. In some embodiments, the report is provided in a format compatible with the device. In some embodiments, the report provides assignments of a reaction mixture and a reaction chamber condition to a reaction chamber of the thermocycling device. In some embodiments, the method further comprising providing the data of reaction chamber conditions to the thermocycling device; and controlling the reaction chamber conditions of an individual reaction chamber of the thermocycling device after the individual reaction chamber is filled with a corresponding reaction mixture based on the report. In some embodiments, device comprises controlling reaction chamber conditions of a first subset of individual reaction chambers, wherein the first subset is less than the plurality of the reaction chambers, and generating an initial result. In some embodiments, the device comprises controlling reaction chamber conditions of a second subset of individual reaction chambers based on a second recommendation, wherein the second recommendation is generated using the initial result. In some embodiments, the device comprises a module for measuring the reaction chamber conditions of an individual reaction chamber during a run. In some embodiments, the device adjusts the reaction chamber conditions of an individual reaction chamber during the run based on the measured reaction chamber conditions. In some embodiments, the device adjusts the recommendation based on the measured reaction chamber conditions. In some embodiments, the device provides a report of the measured reaction chamber conditions.

Provided herein are computer-implemented methods of training an artificial intelligence for a chemical or biological reaction, comprising: obtaining data of a target product, data of reagents, data of reaction chamber conditions, and a quality score of a reaction product; applying, by a computing system, the data to an artificial intelligence; training, by the computing system, the artificial intelligence, wherein training comprises: assigning a connection between one of the data of reagents and one of the data of reaction chamber conditions; assigning a weight to the connection; generating a reward signal from the quality score of the reaction product; and updating the weight based on the reward signal. In some embodiments, training further comprises determining a sequence of connections that provides a reaction product quality score above a threshold score. In some embodiments, the reaction product is a polynucleotide.

Provided herein are computer-implemented methods of generating a recommendation of a design of experiment for a polynucleotide synthesis comprising: obtaining data of a target molecule; applying, by a computing system, the data to a trained artificial intelligence, wherein training of the artificial intelligence comprises: assigning a connection between one of data of reagents and one of data of reaction chamber conditions or between one of data of reagents and another of data of reagents; assigning a weight to the connection; generating a reward signal from a polynucleotide quality score; and updating the weight based on the reward signal; determining, by the computing system, a recommendation of a design of an experiment by the artificial intelligence, wherein determining comprises: determining reagents and reaction chamber conditions to include in the recommendation based on the data of reagents and the data of reaction chamber conditions in the artificial intelligence; and determining a sequence of the connections of the data of reagents and the data of reaction chamber conditions that provides a polynucleotide quality score above a threshold score as the recommendation; generating, by the computing system, a report of the recommendation, wherein the recommendation comprises the data of reagents and the data of reaction chamber conditions from the sequence. In some embodiments, the target molecule is a polynucleotide. In some embodiments, the artificial intelligence comprises machine learning. In some embodiments, the machine learning comprises reinforcement learning. In some embodiments, the reinforcement learning comprises heuristic optimization, wherein the heuristic optimization reduces a number of the sequences. In some embodiments, the methods comprise applying, by the computing system, a dimensionality reduction prior to step of applying, by a computing system, the data to a trained artificial intelligence, wherein training of the artificial intelligence. In some embodiments, the dimensionality reduction comprises clustering of data of oligonucleotides into groups, wherein the groups are based on similar experimental behaviors of the oligonucleotides. In some embodiments, the dimensionality reduction provides an improvement in mapping of sequences, wherein the improvement is characterized by improvement in polynucleotide quality score, computing time, or computing cost. In some embodiments, the data of reagents comprises data of a reagent identification, a reagent volume, and a reagent concentration. In some embodiments, the data of reagents comprises data of a sequence of a nucleic acid molecule, a volume of the nucleic acid molecule, and a concentration of the nucleic acid molecule. In some embodiments, the nucleic acid molecule is an oligonucleotide. In some embodiments, the data of reaction chamber conditions comprises data of a target temperature, a temperature ramp rate, and a time duration at the target temperature. In some embodiments, the data of reaction chamber conditions comprises specified reaction chamber conditions provided before a run. In some embodiments, the data of reaction chamber conditions comprises measured reaction chamber conditions provided after a run. In some embodiments, the polynucleotide quality score provides a level of fidelity of a synthesized polynucleotide sequence compared to a targeted polynucleotide sequence. In some embodiments, training comprises updating a map of connections of the data of the reagents and the data of the reaction chamber conditions based on the data for the oligonucleotides to improve the polynucleotide quality score. In some embodiments, the report is provided in a format compatible with a thermocycling device for polynucleotide synthesis. In some embodiments, the report provides assignments of a reaction mixture and a reaction chamber condition to a reaction chamber of the thermocycling device. In some embodiments, the method further comprises providing the data of reaction chamber conditions to the thermocycling device; controlling the reaction chamber conditions of an individual reaction chamber of the thermocycling device after the individual reaction chamber is filled with a corresponding reaction mixture based on the report. In some embodiments, the device comprises a plurality of individual reaction chambers, wherein the reaction chamber conditions of the individual reaction chamber is capable of being controlled independently from another individual reaction chamber in the device. In some embodiments, the device comprises 96 individual reaction chambers. In some embodiments, the training takes place in an offline mode, wherein the computing system is not connected to a network. In some embodiments, the training takes place in an online mode, wherein the computing system is connected to a network. In some embodiments, the method further comprises communicating the report to a device for polynucleotide synthesis and providing the reaction chamber conditions to the device. In some embodiments, the computing system is within a remote server or an external database remote from a user. In some embodiments, the computing system is within a server or a database local to a user. In some embodiments, the data are obtained by optical character recognition, voice recognition, touchscreen display input, barcode scanning, or user-initiated data input. In some embodiments, the method comprises controlling reaction chamber conditions of a first subset of individual reaction chambers, wherein the first subset is less than the plurality of the reaction chambers, and generating an first result. In some embodiments, the method comprises controlling reaction chamber conditions of a second subset of individual reaction chambers based on a second recommendation, wherein the second recommendation is generated using the first result, and generating a second result. In some embodiments, the second result has a higher polynucleotide quality score than the first result. In some embodiments, the device comprises a module for measuring the reaction chamber conditions of an individual reaction chamber during a run. In some embodiments, the module measures measuring the reaction chamber conditions of an individual reaction chamber in real-time. In some embodiments, the device adjusts the reaction chamber conditions of an individual reaction chamber during the run based on the measured reaction chamber conditions. In some embodiments, the device adjusts the recommendation based on the measured reaction chamber conditions. In some embodiments, the method comprises measuring the deviation of the measured reaction chamber conditions from the specified reaction chamber conditions; correlating the comparison to the polynucleotide quality score; and adjusting the recommendation based on the correlation. In some embodiments, the device provides a report of the measured reaction chamber conditions.

Provided herein are systems, methods, and devices for training an artificial intelligence for a polynucleotide synthesis comprising: at least one processor; and a memory storing instructions that, when executed by the at least one processor, cause the system to perform: obtaining data of a sequence of a target polynucleotide, data of oligonucleotides, data of reagents, data of reaction chamber conditions, and a polynucleotide quality score; applying, by a computing system, the data to an artificial intelligence; training, by the computing system, the artificial intelligence, wherein training comprises: assigning a connection between one of the data of reagents and one of the data of reaction chamber conditions; assigning a weight to the connection; generating a reward signal from the polynucleotide quality score; and updating the weight based on the reward signal.

Provided herein are systems, methods, and devices for generating a recommendation of a design of experiment for a polynucleotide synthesis comprising: at least one processor; and a memory storing instructions that, when executed by the at least one processor, cause the system to perform: obtaining data of a sequence of a target polynucleotide and data of oligonucleotides; applying, by a computing system, the data to a trained artificial intelligence, wherein training of the artificial intelligence comprises: assigning a connection between one of data of reagents and one of data of reaction chamber conditions or between one of data of reagents and another of data of reagents; assigning a weight to the connection; generating a reward signal from a polynucleotide quality score; and updating the weight based on the reward signal; determining, by the computing system, a recommendation of a design of an experiment by the artificial intelligence, wherein determining comprises: determining reagents and reaction chamber conditions include in the recommendation based on the data of reagents and the data of reaction chamber conditions in the artificial intelligence; and determining a sequence of the connections of the data of reagents and the data of reaction chamber conditions that provides a polynucleotide quality score above a threshold score as the recommendation; and generating, by the computing system, a report of the recommendation, wherein the recommendation comprises the data of reagents and the data of reaction chamber conditions from the sequence. In some embodiments, the artificial intelligence comprises machine learning. In some embodiments, the machine learning comprises reinforcement learning. In some embodiments, the systems comprise applying, by the computing system, a dimensionality reduction prior to step of applying, by a computing system, the data to a trained artificial intelligence. In some embodiments, the polynucleotide quality score provides a level of fidelity of a synthesized polynucleotide sequence compared to a targeted polynucleotide sequence. In some embodiments, the training comprises updating a map of connections of the data of the reagents and the data of the reaction chamber conditions based on the data for the oligonucleotides to improve the polynucleotide quality score. In some embodiments, the report is provided in a format compatible with a thermocycling device for polynucleotide synthesis. In some embodiments, the report provides assignments of a reaction mixture and a reaction chamber condition to a reaction chamber of the thermocycling device. In some embodiments, the method further comprises providing the data of reaction chamber conditions to the thermocycling device; controlling the reaction chamber conditions of an individual reaction chamber of the thermocycling device after the individual reaction chamber is filled with a corresponding reaction mixture based on the report. In some embodiments, the device comprises a plurality of individual reaction chambers, wherein the reaction chamber conditions of the individual reaction chamber is capable of being controlled independently from another individual reaction chamber in the device. In some embodiments, the device comprises 96 individual reaction chambers.

Provided herein are methods, systems, and devices for polynucleotide synthesis, comprising: uploading data of a polynucleotide of interest to a computer-based application comprising an artificial intelligence using a computer; receiving a recommendation of a design of experiment, comprising determining, by artificial intelligence from the data of the polynucleotide of interest, reagents, reagent conditions, and reaction chamber conditions for polynucleotide synthesis to generate a polynucleotide with a high fidelity; and providing a recommendation of reagents, reagent conditions, and reaction chamber conditions for polynucleotide synthesis of the polynucleotide of interest; selecting reagent conditions and reaction chamber conditions from the recommendation to use in polynucleotide synthesis; optionally selecting reagent conditions and reaction chamber conditions not provided in the recommendation to use in polynucleotide synthesis, wherein selecting comprises choosing a reagent of interest, conditions for the reagent of interest, and reaction chamber conditions; preparing reaction mixtures from the selected reagent conditions; loading the reaction mixtures to the corresponding reaction chambers based on the recommendation; starting the thermocycling device to perform synthesis of polynucleotide of interest. In some embodiments, the method further comprises assessing the quality of the synthesized polynucleotide products and uploading the quality to the computer-based application. In some embodiments, the data of reagents comprises data of a reagent identification, a reagent volume, and a reagent concentration. In some embodiments, the data of reagents comprises data of a sequence of a nucleic acid molecule, a volume of the nucleic acid molecule, and a concentration of the nucleic acid molecule. In some embodiments, the nucleic acid molecule is an oligonucleotide. In some embodiments, the data of reaction chamber conditions comprises data of a target temperature, a temperature ramp rate, and a time duration at the target temperature.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an internal view of an exemplary thermocycling device.

FIG. 2 shows an example of a rotating actuator for an exemplary thermocycling device.

FIG. 3 shows a detailed view of the core assembly mounted on top of the rotating base in an exemplary thermocycling device.

FIG. 4 shows a detailed view of the power coupling assembly with the contacts in the energized position in an exemplary thermocycling device.

FIG. 5 shows a section view of well sleeve showing installation slot, thermistor, and thermoelectric module in an exemplary thermocycling device.

FIG. 6 shows an exploded view of core assembly showing component parts including cover, threaded well plate, thermoelectric PCB and thermistor PCB in an exemplary thermocycling device.

FIG. 7 shows a view of the dynamic routing mechanism of the signal wires in an exemplary thermocycling device in a load positions and a run position.

FIG. 8 shows a block diagram demonstrating examples of user input methods for experimental runs and how the inputs interface to an online database. Once uploaded, examples of the process of the machine learning algorithms optimizing the experimental process conditions and sending back the recommended experimental process to device are shown.

FIG. 9 shows an example of high level system architecture for thermal portion of the instrument.

FIG. 10 shows an example of a microprocessor architecture.

FIG. 11 shows an example of well sleeve temperature control architecture.

FIG. 12 shows an example of a flowchart of the reinforcement learning process.

FIG. 13A shows an example of a reinforcement learning for a positive or a good outcome.

FIG. 13B shows an example of a reinforcement learning for a negative or a bad outcome.

FIG. 14A shows an example of a solution search space for the reinforcement learning process.

FIG. 14B shows an example of a solution search space for the reinforcement learning process.

FIG. 14C shows an example of in situ experimental optimization.

FIG. 14D shows an example of instrument profile optimization with data collected during operation.

FIGS. 15A-15D shows an example of a flowchart of the reinforcement learning in providing recommended experimental conditions for a polynucleotide synthesis in a web-based application.

FIG. 16 shows an example of a slip ring that transmits power to core assembly of an exemplary thermocycling device.

FIG. 17 shows an example of a polynucleotide synthesis device.

FIGS. 18A-G shows an exemplary web-based interface that is used to control the devices, systems, and methods provided herein.

FIGS. 19A-E shows an exemplary web-based interface for designing the experiment and temperature protocols for nucleotide synthesis.

FIG. 20 shows an exemplary thermoelectric cooler (TEC) board assembly.

FIG. 21 shows an exemplary TEC board and well assembly.

FIG. 22 shows an exemplary TEC board and well assembly.

FIG. 23 shows an exemplary embodiment of the temperature assessment device.

FIG. 24 shows an example of thermograph of wells in an assay plate.

FIG. 25 shows an example of temperature profile over time of individual wells.

FIG. 26 shows an exemplary JavaScript Object Notation (JSON) structure for controlling the device.

### DETAILED DESCRIPTION

Custom gene synthesis, or custom polynucleotide synthesis, provides a powerful tool for research in biology and medicine and for various biotechnology applications. Gene synthesis, or polynucleotide synthesis, typically involves building specially designed oligonucleotides, preparing the oligonucleotides and various reagents in a mixture, and assembling the oligonucleotides using a thermocycler to generate a polynucleotide or a gene. Once the mixture comprising the oligonucleotides and various reagents is placed inside the thermocycler, the mixture is exposed to a temperature profile that is iterated several times. An example of such polynucleotide synthesis is Gibson assembly. One hurdle in polynucleotide synthesis is that assembling complex polynucleotides from oligonucleotides, or nucleic acid molecules, often can be error prone. There is a need for improved methods for custom polynucleotide synthesis, including improving the quality of the synthesized polynucleotide and reducing the time required to generate the polynucleotides.

The quality of a polynucleotide generated by the polynucleotide synthesis may depend on production of the oligonucleotides, choice of reagents, concentrations of reagents or oligonucleotides, or temperature profiles that the mixture is subject to during thermocycling. The quality of the polynucleotide, or the success or failure of the polynucleotide synthesis, may be assessed by running the reacted mixture on an agarose gel or cloning the product into a plasmid and sequencing it.

Often, the custom polynucleotide molecules have errors and are not assembled properly. In order to generate the polynucleotide without an error, the entire polynucleotide synthesis process may be started over with adjustments in the synthesis conditions. The adjustments to new conditions are typically performed by an experienced person who is skilled in the art of custom polynucleotide assembly. The new adjusted conditions sometimes comprise at least one of adding a new reagent, changing a concentration of a reagent in the mixture, or changing the temperature conditions that the mixture is subjected to by the thermocycler. Often, the adjustments to the polynucleotide synthesis processes comprise changing the temperature and/or the length of time at a given temperature that the mixture is subject to by the thermocycler. Many commercially available thermocyclers are limited to less than 8 different temperature reaction chambers. This limits the number of simultaneous polynucleotide synthesis process conditions that can be tested at one time.

In the chaotic laboratory environment, it may be difficult to keep track of the numerous input factors that go into various experiments. These input factors are critical to assessing trends in experiments, but it can be a tedious process for a user to record the details of the step, especially in a research and development environment where intuitive decisions are highly valued. Experimental details can be recorded in laboratory notebooks, or digitally using spreadsheets or other software programs. However, in a lab environment, freeing one's hands from a pipette, or removing a glove to record experimental details may be often put aside until the end of the experiment. This may leave room for error in the records of the experiment.

The devices, methods, and systems provided herein may improve throughput, reduce cost, and provide simple user interface to facilitate collaboration in polynucleotide synthesis. The devices, methods, and systems may comprise a thermocycler comprising a plurality of individual chambers, where the temperature setting of an individual chamber are controlled independently from the temperature setting of another individual chamber, and a system comprising a machine learning for a polynucleotide synthesis for generating a recommendation of a design of experiment for a polynucleotide synthesis. The devices, methods, and systems provided herein can control parameters of individual wells and is small, modular, and easy to maintain. The devices, methods, and systems provided herein may be compatible with laboratory automation, mobile devices, and laboratory information management software (LIMS) to improve throughput, synthesis quality, and reproducibility. Such devices, methods, and systems may improve quality of the polynucleotide product and success of the polynucleotide synthesis by automation and traceability of steps to reduce human errors and by cloud connectivity for distribution of optimized protocols for polynucleotide synthesis. The devices, methods, and systems provided herein may improve the throughput by running multiple protocols with different steps in parallel and using modular architecture that makes it easy to scale. The devices, methods, and systems provided herein may reduce costs by using fewer instruments where each well serves as its own device and has an independent functionality from another well. The devices, methods, and systems provided herein may also reduce costs by reducing labor where the device can fit on a robot deck and is compatible with automated liquid handlers. As a result, such devices, methods, and systems can reduce capital cost, labor cost, space and energy consumption in a laboratory.

Disclosed herein are devices, methods, and systems for automated polynucleotide synthesis comprising a thermocycler device comprising a plurality of individual chambers, where the individual chamber have a capability to control its own temperature setting, and a machine learning for generating a recommendation of experimental conditions for a polynucleotide synthesis. Through practice of disclosure herein, a user inputs a desired polynucleotide to the system comprising the machine learning to generate a recommendation of a design of experiment for a polynucleotide synthesis, comprising experimental conditions. The user can prepare the reaction mixtures using recommended experimental conditions or using their own experimental conditions. The reaction mixtures can undergo polynucleotide assembly in the thermocycler device having a plurality of chambers, where an individual chamber and the reaction mixture in contact with the individual chamber have their own temperature setting. This allows for testing a variety of temperature conditions (i.e. temperatures and duration of the temperatures) for a single reaction mixture. Practice of some part of the disclosure herein achieves a more automated polynucleotide synthesis. Practice of some part of the disclosure herein achieves a more efficient polynucleotide synthesis with higher fidelity, or a more automated polynucleotide synthesis, by the use of the system comprising the machine learning and the device comprising a plurality of individual chambers with its own temperature control. Practice of some part of the disclosure herein provides a faster way to synthesize a polynucleotide having a higher fidelity and determine the conditions that synthesize the polynucleotide having the higher fidelity. In some cases, practice of some part of the disclosure herein reduces process time to less than one day to one week as compared to from around 2 weeks to 6 months.

Provided herein are devices, methods, and systems for thermocycling for polynucleotide synthesis, comprising a plurality of reaction chambers, wherein a temperature setting of an individual reaction chamber can be controlled independent from a temperature setting of another individual reaction chamber. The device disclosed herein may interface with an online database comprising an artificial intelligence, wherein the artificial intelligence generates a report of a recommendation to synthesize a polynucleotide of interest having a high fidelity and wherein the recommendation comprises reagent conditions and reaction chamber conditions. Alternatively or in combination, the device disclosed herein may interface with a web-based interface and an online database. In some embodiment, a user selects and save reagent conditions and reaction chamber conditions in the online database. The web-based interface may control the reaction chamber conditions of the device based on the user selection.

Provided herein are computer-implemented methods of training an artificial intelligence for a chemical or biological reaction, comprising: obtaining data of a target product, data of reagents, data of reaction chamber conditions, and a quality score of a reaction product; applying, by a computing system, the data to an artificial intelligence; training, by the computing system, the artificial intelligence, wherein training comprises: assigning a connection between one of the data of reagents and one of the data of reaction chamber conditions; assigning a weight to the connection; generating a reward signal from the quality score of the reaction product; and updating the weight based on the reward signal.

Provided herein are computer computer-implemented methods of generating a recommendation of a design of experiment for a polynucleotide synthesis comprising: obtaining data of a target molecule; applying, by a computing system, the data to a trained artificial intelligence, wherein training of the artificial intelligence comprises: assigning a connection between one of data of reagents and one of data of reaction chamber conditions or between one of data of reagents and another of data of reagents; assigning a weight to the connection; generating a reward signal from a polynucleotide quality score; and updating the weight based on the reward signal; determining, by the computing system, a recommendation of a design of an experiment by the artificial intelligence, wherein determining comprises: determining reagents and reaction chamber conditions to include in the recommendation based on the data of reagents and the data of reaction chamber conditions in the artificial intelligence; and determining a sequence of the connections of the data of reagents and the data of reaction chamber conditions that provides a polynucleotide quality score above a threshold score as the recommendation; generating, by the computing system, a report of the recommendation, wherein the recommendation comprises the data of reagents and the data of reaction chamber conditions from the sequence. The methods disclosed herein further comprises providing the data of reaction chamber conditions to the thermocycling device; and controlling the reaction chamber conditions of an individual reaction chamber of the thermocycling device after the individual reaction chamber is filled with a corresponding reaction mixture based on the report.

Provided herein are systems for training an artificial intelligence for a polynucleotide synthesis comprising: at least one processor; and a memory storing instructions that, when executed by the at least one processor, cause the system to perform: obtaining data of a sequence of a target polynucleotide, data of oligonucleotides, data of reagents, data of reaction chamber conditions, and a polynucleotide quality score; applying, by a computing system, the data to an artificial intelligence; training, by the computing system, the artificial intelligence, wherein training comprises: assigning a connection between one of the data of reagents and one of the data of reaction chamber conditions; assigning a weight to the connection; generating a reward signal from the polynucleotide quality score; and updating the weight based on the reward signal.

Provided herein are systems for generating a recommendation of a design of experiment for a polynucleotide synthesis comprising: at least one processor; and a memory storing instructions that, when executed by the at least one processor, cause the system to perform: obtaining data of a sequence of a target polynucleotide and data of oligonucleotides; applying, by a computing system, the data to a trained artificial intelligence, wherein training of the artificial intelligence comprises: assigning a connection between one of data of reagents and one of data of reaction chamber conditions or between one of data of reagents and another of data of reagents; assigning a weight to the connection; generating a reward signal from a polynucleotide quality score; and updating the weight based on the reward signal; determining, by the computing system, a recommendation of a design of an experiment by the artificial intelligence, wherein determining comprises: determining reagents and reaction chamber conditions include in the recommendation based on the data of reagents and the data of reaction chamber conditions in the artificial intelligence; and determining a sequence of the connections of the data of reagents and the data of reaction chamber conditions that provides a polynucleotide quality score above a threshold score as the recommendation; and generating, by the computing system, a report of the recommendation, wherein the recommendation comprises the data of reagents and the data of reaction chamber conditions from the sequence.

Provided herein are methods for polynucleotide synthesis, comprising: uploading data of a polynucleotide of interest to a computer-based application comprising an artificial intelligence using a computer; receiving a recommendation of a design of experiment, comprising determining, by artificial intelligence from the data of the polynucleotide of interest, reagents, reagent conditions, and reaction chamber conditions for polynucleotide synthesis to generate a polynucleotide with a high fidelity; and providing a recommendation of reagents, reagent conditions, and reaction chamber conditions for polynucleotide synthesis of the polynucleotide of interest; selecting reagent conditions and reaction chamber conditions from the recommendation to use in polynucleotide synthesis; optionally selecting reagent conditions and reaction chamber conditions not provided in the recommendation to use in polynucleotide synthesis, wherein selecting comprises choosing a reagent of interest, conditions for the reagent of interest, and reaction chamber conditions; preparing reaction mixtures from the selected reagent conditions; loading the reaction mixtures to the corresponding reaction chambers based on the recommendation; and starting the thermocycling device to perform synthesis of polynucleotide of interest. The methods disclosed herein further comprise assessing the quality of the synthesized polynucleotide products and uploading the quality to the computer-based application.

Disclosed herein is a thermocycling device having a plurality of reaction chambers, where the temperature setting of an individual chamber can be independently controlled. The device provides a user up to a plurality of reaction chambers with its own independent experimental conditions to assemble a polynucleotide. Alternatively or in combination, the device interfaces with an online database where specialized artificial intelligence (AI) algorithms automatically optimize the temperature conditions for each reaction chamber based upon the custom polynucleotide of interest and the reagents for the experiments. In some embodiments, the AI algorithm comprises machine learning (ML) algorithm. In some embodiments, the ML algorithm comprises reinforcement learning (RL) algorithm.

In an exemplary embodiment, the device serves as the main interface to the online database. The user can input any or all factors that are part of the experiment using manual input (via touchscreen display), machine readable barcode, optical character recognition or voice instruction. Alternatively or in combination, the user can input the factors affecting the experiment using a web-based application for the online database from a computer in order to plan the polynucleotide synthesis experiment. In some embodiments, the computer is separate from the device and is not connected directly to the thermocycling device. The computer may be connected to the device by internet. In some embodiments, the computer is connected to the thermocycling device. In some embodiments, the user interface is connected to the thermocycling device or a part of the thermocycling device. Once the experimental factors are selected, the machine learning algorithms search through the online database to select the optimal conditions to run the polynucleotide synthesis experiment. The web-based application can guide the user through the process of designing an experiment, including but not limited to input of polynucleotide of interest, selection of factors recommended by the online database, and addition of additional factors from the user. The chosen experimental conditions can be uploaded to the thermocycling device. Alternatively or in combination, the chosen experimental conditions can be provided to the user as a report, a display on a screen, or an audible verbal instruction through a speaker. The experimental conditions may comprise a type reagent to add, a volume of the reagent, a concentration of the reagent, and instructions on which individual reaction chamber to add the reagent.

The device can subject the loaded reaction mixture in individual reaction chamber to one of the optimized process conditions. The device can perform a plurality of individual experimental conditions in a single run as the temperature setting of the individual reaction chamber can be controlled independently.

In some embodiments, the devices, methods, and systems for polynucleotide synthesis comprises a single device capable of miniaturized liquid handling, multi-channel thermocycling, and polynucleotide sequencing. In some embodiments, the liquid handling component prepares multiple mixtures comprising different combinations of oligonucleotides, primers, and reagents at various concentrations. In some embodiments, the devices, methods, and systems for polynucleotide synthesis runs multiple temperature protocols based upon recommendations from the AI engine in parallel in multiple individual wells. In some embodiments, the resulting mixtures undergo through library preparation on the thermocycling and liquid handling components of the device to synthesize the target polynucleotides.

In some embodiments, at least one of the wells has resulting polynucleotide products having an average of 100% accuracy to the target polynucleotide. In some embodiments, at least one of the wells has resulting polynucleotide products having at least an average of 99% accuracy to the target polynucleotide. In some embodiments, at least one of the wells has resulting polynucleotide products having at least an average of 98% accuracy to the target polynucleotide.

The devices, systems, and methods described herein provide various advantages. In some embodiments, the devices, systems, and methods described herein allow for easier comparison of different protocols within a single run instead of using multiple thermocycler. In some embodiments, the devices, systems, and methods described herein allow for easier sharing and comparison of experimental protocols amongst the users. In some embodiments, the devices, systems, and methods described herein allow for saving of temperature protocols and input process data electronically that is easily accessible by the users and their collaborators.

### Thermocycling Device

The thermocycler device as disclosed herein comprises a rotating baseplate, a motor, a rotating actuator, a microprocessor, thermoelectric modules, thermistors (temperature sensors), a heated lid, a heat sink fan, a power coupling assembly, and a touchscreen display. Optionally, the device further comprises at least one of an online database, a camera, and a microphone. The thermocycling device is also referred herein as thermocycler or thermal cycler.

In some embodiments, the devices, methods, and systems provided herein comprise an integrated microfluidic workflow that monitor and control various process variables. In some embodiments, the various process variables include but are not limited to temperature, volume, time, optical density, or optical characteristics. In some embodiments, the device is a small, high throughput thermocycling device with multichannel control. In some embodiments, the device can combine with existing microfluidic liquid handling and quality control technology. In some embodiments, the device comprises sensors and control methods to regulate, monitor and control various process variables. In some embodiments, the devices, methods, and systems provided herein allow for flexible, reconfigurable workflows.

Provided herein are devices, methods, and systems having a capability to control the functionality of the individual wells independently where an individual well has the functionality of an independent instrument. The independently controlled functionality is achieved by embedded heating, cooling, and sensing mechanism for each individual well. In some embodiments, the devices, methods, and systems achieves 100° C. temperature difference between neighboring wells. In some embodiments, the devices, methods, and systems have temperature accuracy to 0.1° C.

In some embodiments, the devices, methods, and systems provided herein comprise an automated lid for automatic integration. In some embodiments, the devices, methods, and systems comprise samples and reagents that are barcoded and tracked by barcodes, where a barcode is specific to a sample or a reagent. In some embodiments, the devices, methods, and systems comprise a cloud-based or walk up operation.

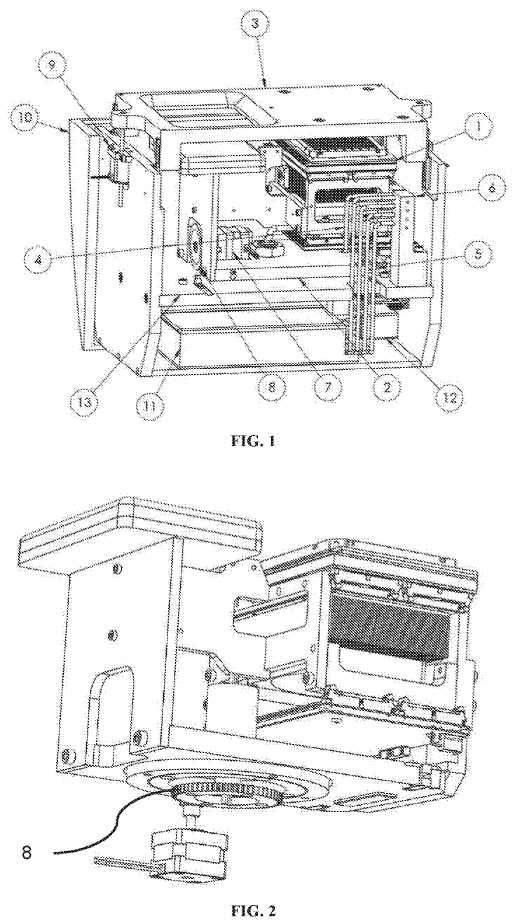

An example of the disclosure provided herein is illustrated in FIG. 1. FIG. 1 shows an internal view of an exemplary thermocycling device. The external housing 10 of the thermocycling device houses the various elements described herein and provides protection for the various elements. The device comprises a lid actuator 9 that can move the heated lid 3 up and down in a vertical direction. In some embodiments, the lid actuator 9 can move the heated lid 3 in the multiple directions. The device comprises a lid actuator 9 that can move the heated lid 3 up and down in a vertical direction relative to the core assembly. In some embodiments, the lid actuator 9 can move the heated lid 3 in the multiple directions relative to the core assembly. The device comprises a lid actuator 9 that can move the heated lid 3 up and down in a vertical direction relative to the external housing. In some embodiments, the lid actuator 9 can move the heated lid 3 in the multiple directions relative to the external housing. Inside the external housing 10 and heated lid 3, the device comprises a core assembly 1, a rotating base plate 2, a moving power cable assembly 4, a fixed power cable assembly 5, a power coupling assembly 6, a baseplate motor 7, a baseplate rotating actuator 8, a DC power supply 11, a power input module 12, and a fixed based plate 13. In some embodiments, the DC power supply 11 is located at the bottom of the device. In some embodiments, the DC power supply 11 is located at a side of the device. In some embodiments, the DC power supply 11 is located adjacent to the power input module 12. In some embodiments, the core assembly 1, the rotating base plate 2, the power coupling assembly 6, the baseplate motor 7, and the baseplate rotating actuator 8 are located above the fixed base plate 13. In some embodiments, the core assembly is located just underneath the heated lid 3. In some embodiments, the baseplate motor 7 and the baseplate rotating actuator 8 move and/or rotate the rotating base plate 2. In some embodiments, the core assembly 1 is mounted on top of the rotating base plate 2. In some embodiments, the power coupling assembly 6 is adjacent to the core assembly 1.

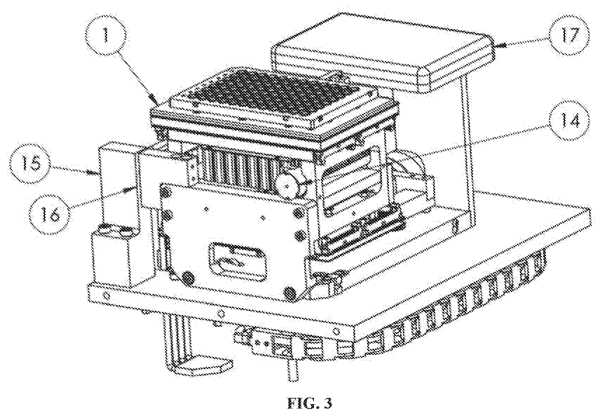

A detailed view of the core assembly mounted on top of the rotating base in an exemplary thermocycling device is shown in FIG. 3. In some embodiments, the device further comprises well plate cover 17, a heat exchanger 14, and power coupling assembly 6, 15, 16. In some embodiments, the core assembly 1 is a top the heat exchanger 14. In some embodiments, the plate cover 17 is completely or partially offset from the core assembly 1. In some embodiments, the well plate cover 17 completely covers a multi-well plate placed on top of the core assembly 1.

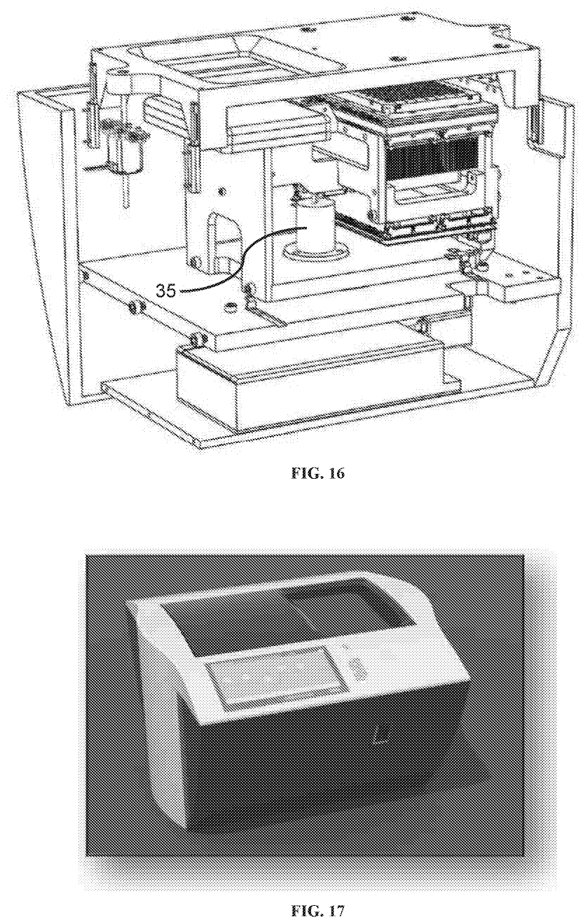

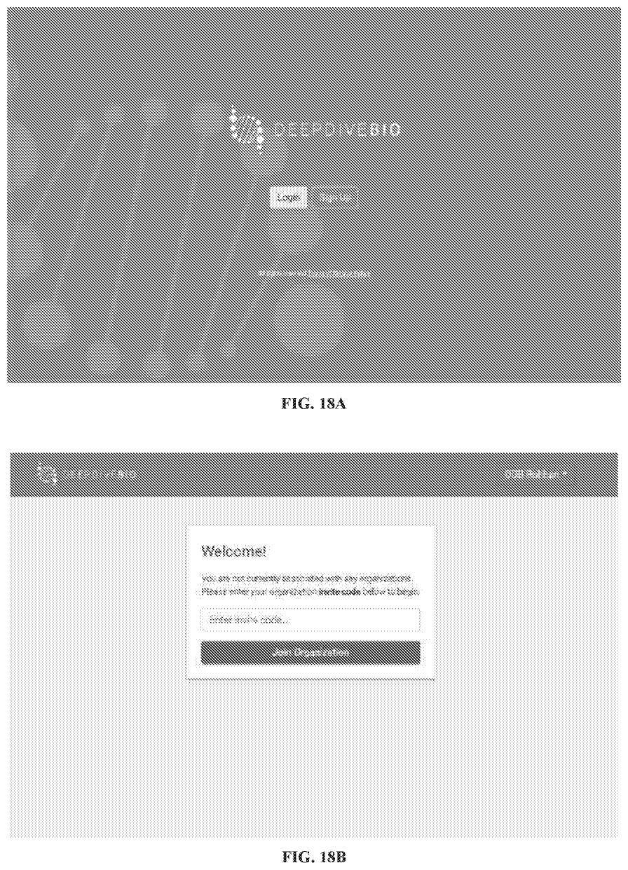

Another example of a rotating actuator 8 for an exemplary thermocycling device is shown in FIG. 2. A view of the core assembly and a slip ring 35 that transmits power to the core assembly of an exemplary thermocycling device is shown in FIG. 16. The slip ring 35 provides a central hole that can accommodate the rotation of the rotating actuator 8 and various elements of the device.

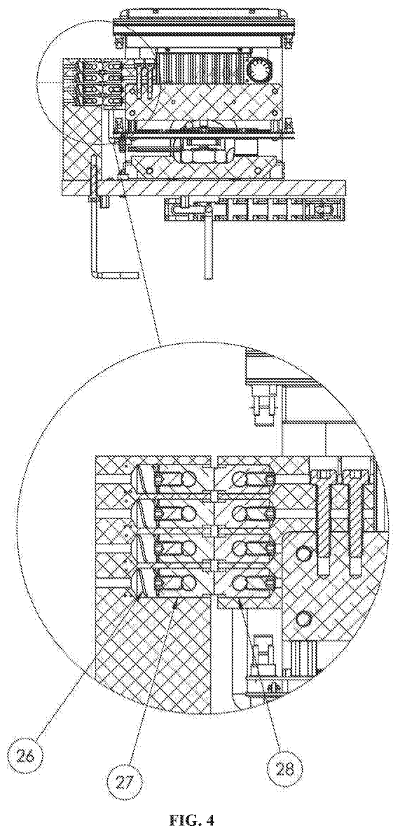

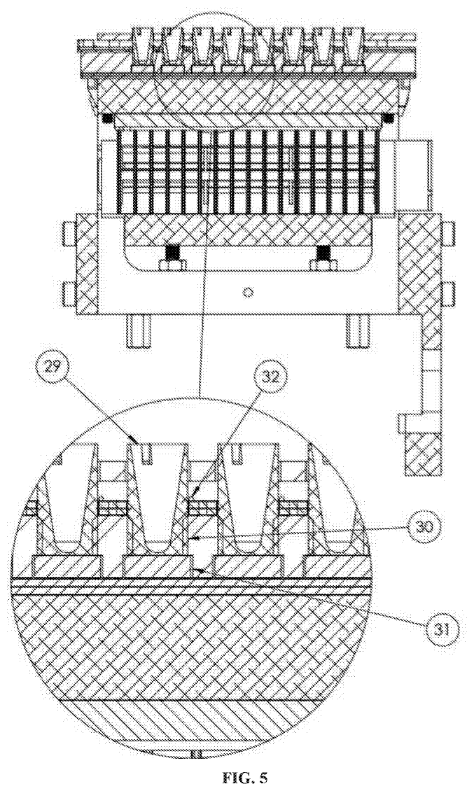

A detailed view of the power coupling assembly with the contacts in the energized position in an exemplary thermocycling device is shown in FIG. 4. In some embodiments, the power coupling assembly 6, 15, 16 comprises an electrode spring 26, a fixed electrode 27, and a moving electrode 28.

A section view of well sleeve showing installation slot, thermistor, and thermoelectric module in an exemplary thermocycling device is shown in FIG. 5. In some embodiments, the device comprises a slotted well sleeve 29, a well sleeve thread 30, a thermoelectric module 31, and a thermistor 32. In some embodiments, the device comprises a plurality of slotted well sleeves 29, where the individual well sleeve comprises a well sleeve thread 30, a thermoelectric module 31, and a thermistor 32. In some embodiments, the slotted well sleeves are located on top of the core assembly 1. In some embodiments, the device has the capability to control the temperature of individual chamber independently from its neighboring chamber. In some embodiments, the thermistor, and thermoelectric module described herein provide the capability to control the temperature of individual chamber independently. In some embodiments, the device comprises 96 chambers. In some embodiments, the device comprises 6, 12, 24, 48, 96, 384, or 1536 chambers. In some embodiments, the device comprises at least 10, 20, 30, 40, 50, 60, 70, 80, 90, 100, 200, 300, 400, or 500 chambers. In some embodiments, a commercially available well plate is placed atop the well sleeve or in place of the well sleeve. In some embodiments, the well sleeve is dimensioned similarly to commercially available plate as to be compatible with various laboratory equipments, including but not limited to manual and automated pipettes.

In some embodiments, the well sleeve is a chamber or a reaction chamber. In some embodiments, a chamber or a reaction chamber is placed inside the well sleeve and the well sleeve surrounds the outside wall of the chamber or well. In some embodiments, the well sleeve comprises materials compatible with polynucleotide synthesis reaction. In some embodiments, the material for well sleeve comprises at least one of polyethylene, polypropylene, PTFE, plastics, and polymers. In some embodiments, the well sleeve comprises a heat conducting material. In some embodiments, the well sleeve comprise comprises heat conducting metal. In some embodiments, the well sleeve comprises aluminum. In some embodiments, the well sleeve comprises stainless steel. In some embodiments, the material for well sleeve is coated to provide a desired feature, including but not limited to increased hydrophilicity, hydrophobicity, low binding, etc.

In some embodiments, the individual well sleeve can hold various volumes. In some embodiments, the individual well sleeve holds at least 0.01 ml, at least 0.02 ml, at least 0.03 ml, at least 0.04 ml, at least 0.05 ml, at least 0.06 ml, at least 0.07 ml, at least 0.08 ml, at least 0.09 ml, at least 0.1 ml, at least 0.2 ml, at least 0.3 ml, at least 0.4 ml, at least 0.5 ml, at least 0.6 ml, at least 0.7 ml, at least 0.8 ml, at least 0.9 ml, or at least 1.0 ml. In some embodiments, the individual well sleeve holds about 0.01 ml, 0.02 ml, 0.03 ml, 0.04 ml, 0.05 ml, 0.06 ml, 0.07 ml, 0.08 ml, 0.09 ml, 0.1 ml, 0.2 ml, 0.3 ml, 0.4 ml, 0.5 ml, 0.6 ml, 0.7 ml, 0.8 ml, 0.9 ml, or 1.0 ml. In some embodiments, the individual well sleeve holds no more than 0.01 ml, 0.02 ml, 0.03 ml, 0.04 ml, 0.05 ml, 0.06 ml, 0.07 ml, 0.08 ml, 0.09 ml, 0.1 ml, 0.2 ml, 0.3 ml, 0.4 ml, 0.5 ml, 0.6 ml, 0.7 ml, 0.8 ml, 0.9 ml, or 1.0 ml.

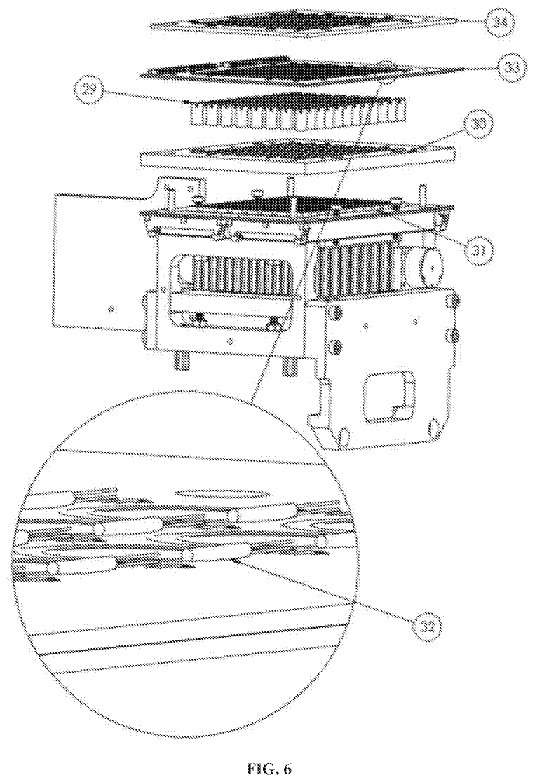

An exploded view of core assembly showing component parts including cover, threaded well plate, thermoelectric PCB and thermistor PCB in an exemplary thermocycling device is shown in FIG. 6. The modular core assembly may provide flexibility in the well sleeve dimension and volume. In some embodiments, the core assembly comprises a slotted well sleeve 29, a threaded well baseplate 30, a thermoelectric module 31, a thermistor 32, a thermistor PCBA (printed circuit board assembly), and a cover plate 34. In some embodiments, the thermoelectric module 31 is mated to PCB (printed circuit board). In some embodiments, the thermoelectric module 31 provides a base for the core assembly. In some embodiments, the core assembly has from the bottom to the top, the thermoelectric module 31, the threaded well baseplate 30, the slotted well sleeves 29, the thermistor PCBA 33, and the cover plate 34 stacked on top of each other. In some embodiments, the components of the core assembly are aligned and assembled together before performing a polynucleotide synthesis. In some embodiments, a plurality of thermistors 32 is on the thermistor PCBA. In some embodiments, the modular core assembly allows for changes to configuration. In some embodiments, the modular core assembly comprises a chassis that supports plug and play. In some embodiments, addition of a chassis expands the number of channels from the number of channels of a base thermocycler. In some embodiments, addition of a chassis expands from the 16-channels of a base thermocycler to 1536 channels.

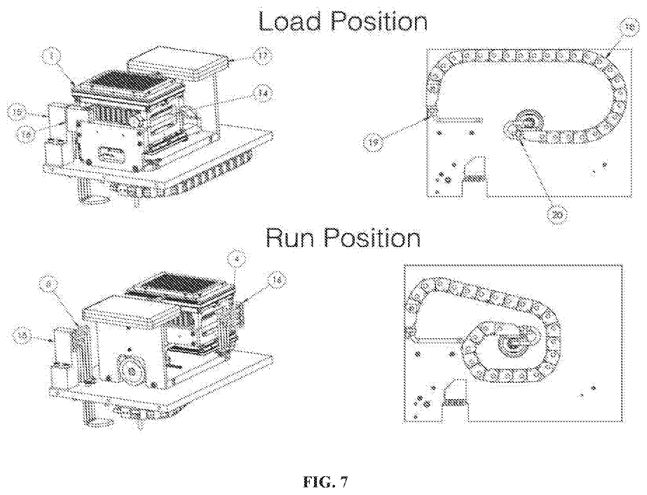

Top and bottom views of the dynamic routing mechanism of the signal wires in an exemplary thermocycling device in a load positions and a run position is shown in FIG. 7. In some embodiments, the base plate rotating actuator 8 and the base plate motor 7 are operatively coupled with a base plate drive belt that moves the rotating baseplate 2 from a load position to a run position. In some embodiments, the drive belt is a multi-rib belt. In moving the rotating baseplate 2 from a load position to a run position, the core assembly moves to switch positions with the well plate cover. In some embodiments, the place the core assembly is moved from underneath an opening in the heated lid 3 to away from the opening. In some embodiments, the place the core assembly is moved to the opening in the heated lid 3. In some embodiments, the device further comprises a convective cooling assembly 14, a flexible cable guide 18, a fixed portion of the cable guide 19, and moving portion of the cable guide 20.

An example of high level system architecture for thermal portion of an exemplary thermocycling device is shown in FIG. 9. In some embodiments, the thermal portion of the device comprises a USB connector, USB to UART bridge controller, a microcontroller, a motor, a heat sink, a fan, a heater, a non-volatile memory, a AC/DC power supply, a fuse protection, a DC-DC regulator, a thermoelectric cooler (TEC) driver, a TEC, and a negative temperature coefficient (NTC) thermistor.

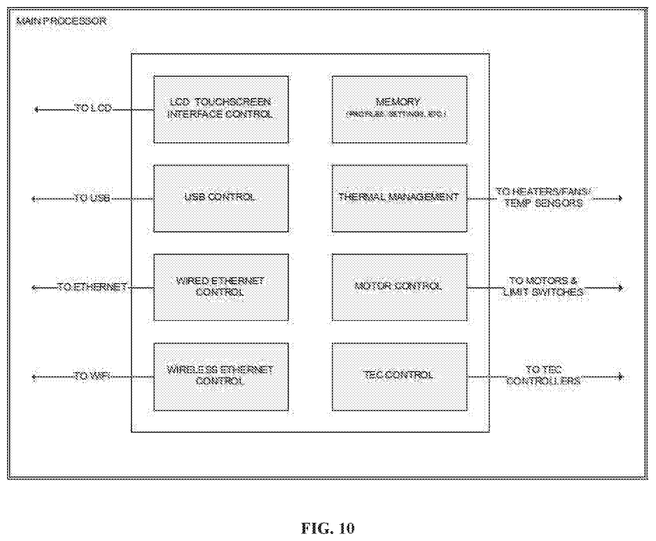

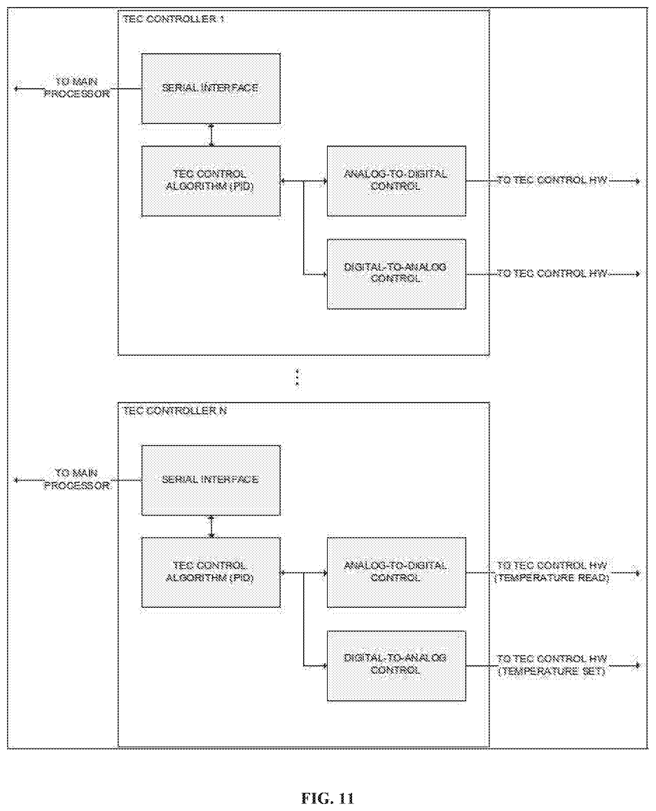

An example of a microprocessor architecture is shown in FIG. 10. In non-limiting embodiments disclosed herein, the main processor comprises at least one of a touchscreen interface, a USB control, a wired Ethernet control, a wireless Ethernet control, a memory, a thermal management control, a motor control, and a TEC control. In some embodiments, the touchscreen interface connects with a screen. In some embodiments, the USB control connects to the USB connector. In some embodiments, the wired Ethernet control connects to the Ethernet. In some embodiments, the wireless Ethernet control connects to WiFi. In some embodiments, the memory provides information on the profiles and setting. In some embodiments, the thermal management control connects to at least one of heaters, fans, and temperature sensors. In some embodiments, the motor control connects to the motors and limit switches. In some embodiments, the TEC control connects to TEC controllers.

An example of well sleeve temperature control architecture of an exemplary thermocycling device is shown in FIG. 11. In some embodiments, a thermoelectric cooler (TEC) controller for well sleeve temperature control comprises a serial interface, a TEC control algorithm (PID), an analog-to-digital control, and a digital-to-analog control. In some embodiments, the serial interface is in communication to the main processor and is in communication with the TEC control algorithm (PID). In some embodiments, the TEC control algorithm (PID) is in communication with the serial interface, the analog-to-digital control, and the digital-to-analog control. In some embodiments, the analog-to-digital control is in communication to TEC control hardware for reading the temperature. In some embodiments, the digital-to-analog control is in communication to TEC control hardware for setting the temperature. In some embodiments, the device comprises a plurality of TEC controllers. In some embodiments, the individual TEC controller corresponds to an individual chamber or well sleeve.

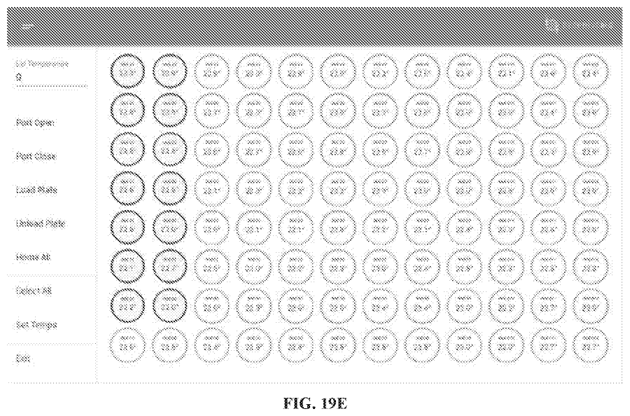

In some embodiments, the thermocycling device comprises a thermoelectric cooler (TEC) board and heat sink to independently control the temperatures of individual chambers. FIG. 20 show an exemplary TEC board shown upside down. The rigid card backing material 201 provides support for multiple thermal adhesive pads 202, which are regularly spaced apart. In some embodiments, the spot of thermal adhesive pads are spaced similarly to the wells of a standard multi-well plate. The thermal adhesive pads 202 are covered by a low tack adhesive 203 for protection during assembly of the TEC board. The thermal adhesive pads 202 are applied to the heat sink 204 before being placed on to the base plate 205.

In an exemplary embodiment, the TEC board assembly is assembled by applying the thermal adhesive pad to the heat sink before placing the TEC board. The TEC board is then placed on to the base plate, and the adhesive sticker protecting the TEC board is removed. The TEC board assembly is mated with the well assembly and secured with screws. A thermistor board is placed onto the mated TEC board-well assembly, and thermal adhesive is applied to each thermistor to bond it to each well. A silicone pad is placed over the assembly. A fiberglass protective cover is further placed on top and secured with screws.

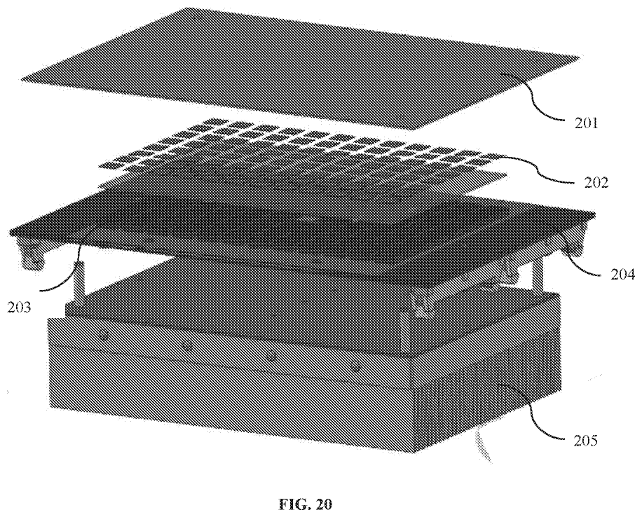

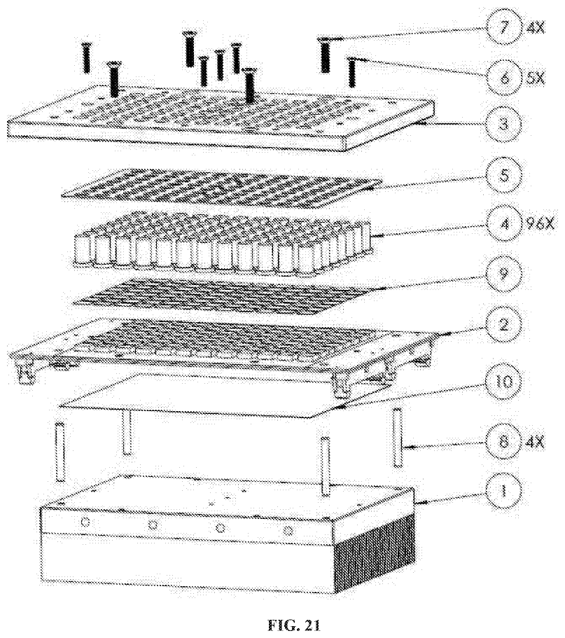

In some embodiments, the thermocycling device comprises a TEC board and well assembly to independently control the temperatures of individual chambers. FIG. 21 shows an exemplary TEC board and well assembly. This assembly comprises a heat sink 1, PCBA, TEC PCB 2, well holder 3, individual wells 4, well pressure mat 5, screws 6 and 7, pin and dowels 8, TEC PCBA thermal adhesive 9, and heat sink thermal adhesive 10.

In an exemplary embodiment, the TEC board and well assembly is assembled by pressing the dowel pins 8 into the heat sink 1 to a predefined height. Apply heatsink adhesive 10 to heatsink 1 in the predefined location. Remove adhesive backing. Adhere TEC PBC 2 to heat sink 1 in a predefined orientation. Press evenly over the entire surface to ensure even adhesion. Attach TEC PCBA adhesive 9 to the top surface of each TEC of the TEC PCB. Leave adhesive backing in place. Assemble well holder 3, silicone mat 6 and all the individual wells 4 in an inverted orientation. Remove TEC adhesive backing from the TEC PCB and bond the PCB with heat sink to the components from the previous step while they remain inverted. Apply even pressure across entire surface area of heat sink bottom to ensure the TEC adhesive bonds to the well bottoms. While holding the bonded components from the previous step together, turn over and install the screws.

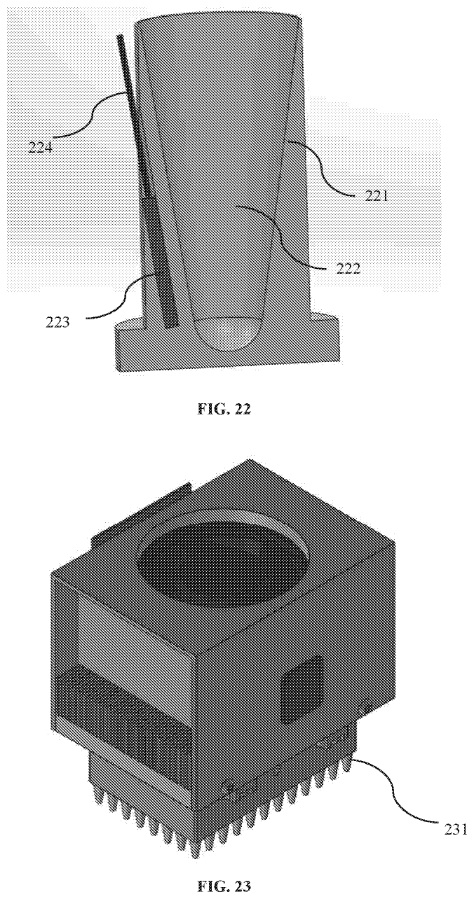

Alternatively or in combination, in some embodiments, the thermocycling device comprises a TEC board and well assembly to independently control the temperatures of individual chambers. In some embodiments, the thermistor active material is integrated into the individual well itself. In some embodiments, the integrated design circumvents the need to apply thermal adhesive and bond the thermistor to each well. FIG. 22 shows exemplary TEC board and well assembly where the thermistor active material is integrated into individual wells. This assembly comprises the chamber wall 221, the chamber or well 222, thermistor active material 223, and wires 224 connecting the thermistor active material to the main board. In some embodiments, the well has a cutout hole on its outside wall to hold the thermistor active material in place. This close location allows the thermistor active material to quickly and efficiently alter the temperature of the well. In some embodiments, the thermistor active material is in contact with the outside wall of the well. In some embodiments, the thermistor active material is potted into a cutout in the outside wall of the well. In some embodiments, the thermistor active material is placed substantially along the height of the well. In some embodiments, the thermistor active material is placed substantially parallel to the inner wall of the well. As shown in FIG. 22, the well has a tapered end having a small diameter at the bottom than the top but can be cylindrical with the same or substantially same diameter throughout its height.

In some embodiments, the spot of thermal adhesive pads have a smaller area than the average diameter of a well of a standard multi-well plate. In some embodiments, the spot of thermal adhesive pads have a smaller area than the average diameter of a well of a standard multi-well plate by at least 10%, 20%, 30%, 40%, or 50%. In some embodiments, the spot of thermal adhesive pads have a smaller area than the average diameter of a well of a standard multi-well plate by no more than 10%, 20%, 30%, 40%, or 50%. In some embodiments, the spot of thermal adhesive pads have a larger area than the average diameter of a well of a standard multi-well plate. In some embodiments, the spot of thermal adhesive pads have a larger area than the average diameter of a well of a standard multi-well plate by at least 10%, 20%, 30%, 40%, or 50%. In some embodiments, the spot of thermal adhesive pads have a larger area than the average diameter of a well of a standard multi-well plate by no more than 10%, 20%, 30%, 40%, or 50%.

In some embodiments, a well sleeve has a larger area than the average diameter of a well of a standard multi-well plate. In some embodiments, a well sleeve has a larger area than the average diameter of a well of a standard multi-well plate by at least 10%, 20%, 30%, 40%, or 50%. In some embodiments, a well sleeve has a larger area than the average diameter of a well of a standard multi-well plate by no more than 10%, 20%, 30%, 40%, or 50%. In some embodiments, the well sleeves in a well assembly have the same dimensions. In some embodiments, the well sleeves in a well assembly have different dimensions. In some embodiments, the well sleeves in a well assembly have more than one diameter. In some embodiments, the well sleeves in a well assembly have more than one height.

Provided herein are devices, systems, and methods to assess and calibrate the temperature control of individual wells. In some embodiments, a temperature assessment device is a calibration tool to assess and calibrate the temperature control of individual wells. In some embodiments, the temperature assessment device fits into a single individual well to calibrate the temperature control of the individual wells over time. In some embodiments, the temperature assessment device comprises a male version of an individual well. In some embodiments, the male version is substantially an inverse casting of the well space within the individual well. In some embodiments, the male version fits closely to the inner wall of the individual wall. In some embodiments, the temperature assessment device comprises a plurality of the male versions. In some embodiments, the temperature assessment device comprises a plurality of the male versions for a 96 well plate. FIG. 23 shows an exemplary embodiment of the temperature assessment device with a male version 231 of the wells. In some embodiments, the temperature assessment device comprises a base, a heat sink, a thermoelectric module, and a RTD. In some embodiments, the temperature assessment device comprises a thermal tape, a thermal gap pad, screws, an enclosure, and a case fan. In some embodiments, the temperature assessment device comprises a circuit board, a microcontroller, and a TEC control board driver. In some embodiments, the temperature assessment device controls the steady state temperature of it very accurately (<0.05° C.). In some embodiments, the temperature assessment device increments the steady state temperature across the operating temperature range. In some embodiments, the operating temperature range is 0-100° C. In some embodiments, the (relative) voltage/measured temperatures in the control circuit are recorded and compared to the (absolute) steady state temperature of the tool. The data is analyzed by fitting a fourth order polynomial for the relative temperatures/voltages as compared to the absolute temperature. This analysis is applied as a correction factor in the software for the devices, systems, and methods described herein.

The temperature assessment device can use various code and steps to calibrate the temperature control of the individual wells. In some embodiments, the temperature assessment device uses a pseudo code. In some embodiments, the process is automated between the temperature assessment device and the thermocycler unit. In some embodiments, a user selects to calibrate on the thermocycler device. This directs the temperature assessment device to begin and start making set point of the temperature assessment device starting temperature. In each cycle, using feedback from the RTD of the temperature assessment device, the temperature assessment device controls the TEC amplifier until a steady state is reached at the set point. In some embodiments, the steady state is reached when the set point temperature varies less than 0.05° C. over a 10 second time interval. Once the steady state is reached, the temperature assessment device begins reading the measured temperatures from the TEC control board and saves the measured temperatures and the set point temperatures. The temperature assessment device begins increasing the set point by a predetermined increment and the cycle is repeated to set temperature, reach steady state, and take the measured temperature until the maximum temperature is reached. When the maximum temperature is reached, the thermocycler communicates to the temperature assessment device to cut the power to the TEC amplifier to cool down the temperature assessment device. Then, the measured temperature data are used to fit a fourth order polynomial for each of the wells. In some embodiments, the measured temperature data are used to fit to various non-linear relationships for each of the wells. Using the coefficients from the non-linear fit, the thermocycler calculates the actual temperature as compared to the measured temperatures from the TEC control board.

### User Interface and Artificial Intelligence for Experiment Design

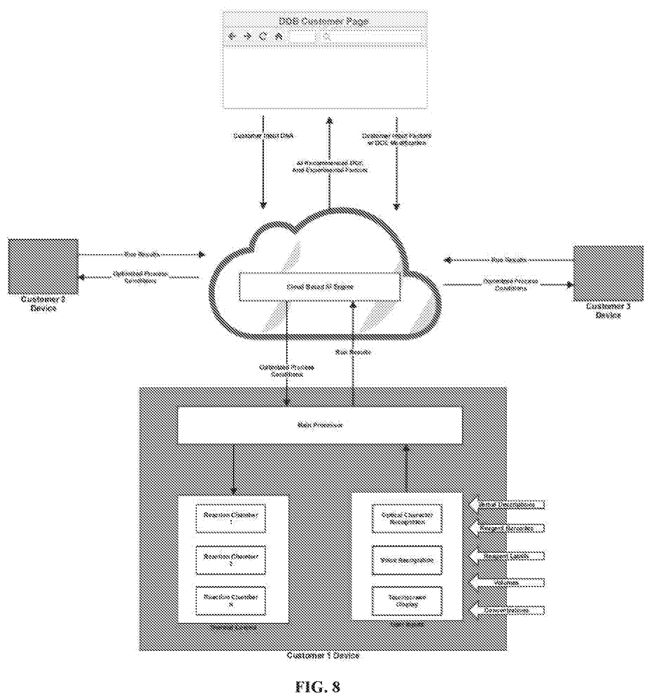

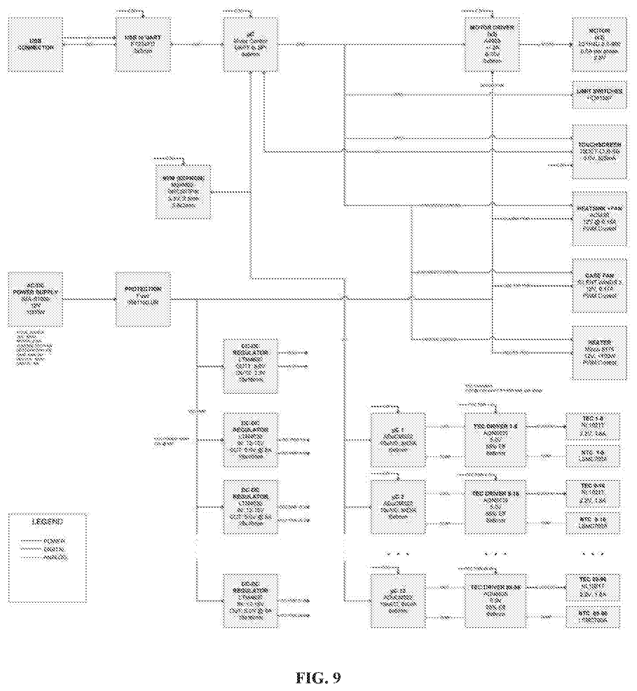

A block diagram demonstrating examples of user input methods for experimental runs and how the inputs interface to an online database is shown in FIG. 8. Once uploaded, examples of the process of the machine learning algorithms optimizing the experimental process conditions and sending back the recommended experimental process to device are shown. In some embodiments, an online database is provided on a remote server with the machine learning algorithms as disclosed herein. In some embodiments, a customer device comprises a main processor, a plurality of reaction chambers with individual reaction chamber having its own thermal control, and a receiver of user inputs. In some embodiments, the receiver of user inputs comprises at least one of optical character recognition, voice recognition, and touchscreen displays. In some embodiments, the user input comprises at least one of verbal descriptions, reagent barcodes, reagent labels, reagent volumes, and reagent concentrations. In some embodiments, the customer device is in communication with the online database, where the online database provides to the customer device a recommendation of optimized process conditions and the customer device provides experimental factors and run results to the online database. In some embodiments, a plurality of customer devices is in communication with the online database. In some embodiments, the communication between the customer device and the online database is wireless. In some embodiments, the communication between the customer device and the online database is wired. Alternatively or in combination, the user can access the online database using a web-based application connected to the online database from a computer. In some embodiments, the computer is not a part of the device. In some embodiments, the computer is connected to the device. Using the web-based application, the user can provide the input polynucleotide (i.e. input DNA) to the online database. The online database can provide an AI recommended design of experiment and experimental factors, which can be displayed to the user using the web-based application. Then, the user can select the experiment factors and/or modify the design of experiment that can be used in the polynucleotide synthesis experiment.

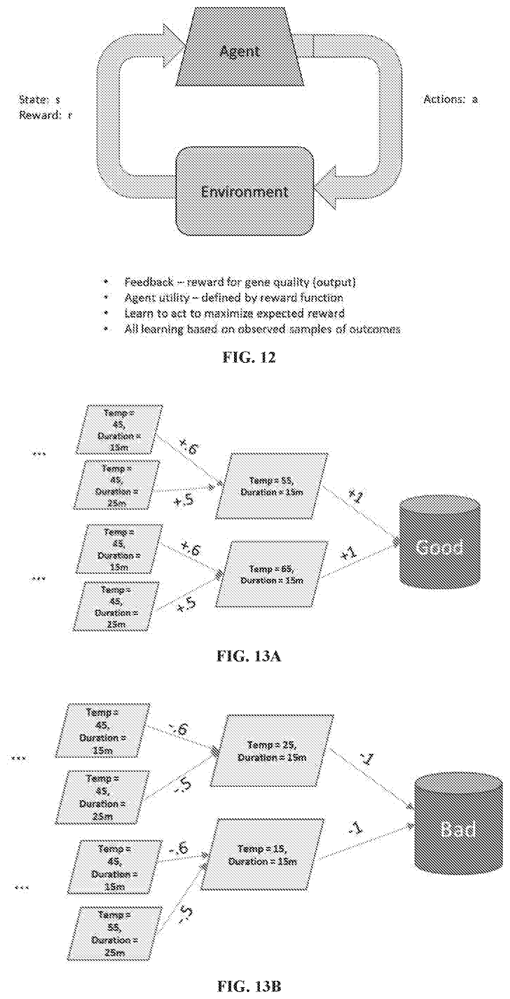

An example of a flowchart of the reinforcement learning process is shown in FIG. 12. In some embodiments, the reinforcement learning comprises an agent and an environment, where the agent provides an action (a) on the environment and the environment provides a feedback of state (s) and a reward (r). In some embodiments, the feedback is a reward for polynucleotide quality. In some embodiments, utility of an agent is defined by the reward function. In some embodiments, the reinforcement learning algorithm learns to act to maximize the expected reward. In some embodiments, the learning is based on observed samples of outcomes.

The reinforcement learning process can be performed offline or online. In some embodiments, the offline learning facilitates learning from a large dataset or learning from data in batches. In some embodiments, online learning facilitates a more real-time feedback, where an experiment can be performed, feedback of the experimental results can be provided to the reinforcement learning algorithm, and the reinforcement learning algorithm can adapt based on the reward signal for the experimental results. In some embodiments, the online learning provides the capability to generate new data and control experiments. In some embodiments, the online learning may be more expensive per experiment because the online learning actuates the environment and uses reagents. In some embodiments, the online learning provides a link to design of experiments (DOE) as well as heuristic optimizations, which can reduce total search space of possible action sets and the processing time.

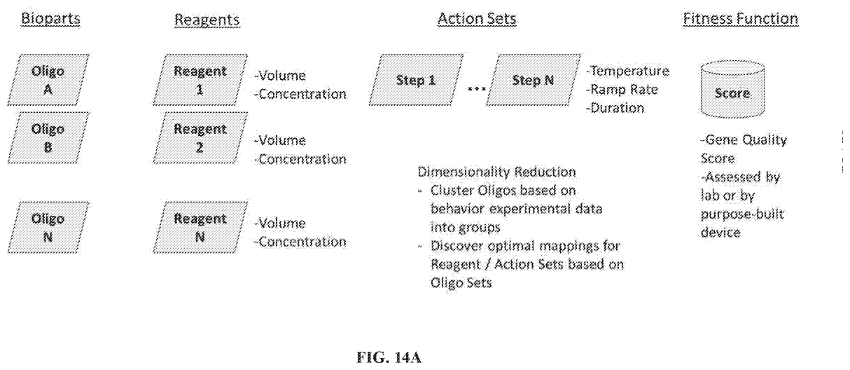

An example of a reinforcement learning for a positive or a good outcome is shown in FIG. 13A and for a negative or a bad outcome is shown in FIG. 13B. In various embodiments of reinforcement learning as disclosed herein, the reward signal is propagated back and weights between each action/state item are updated. As a result of the reinforcement learning, the system eventually learns action/state sequences (action sets) for optimal control based on configured inputs (constraints). FIG. 13A shows the reward of positive weights that are propagated backwards for the various temperature and duration conditions used to generate a polynucleotide with a high fidelity. FIG. 13B shows the backpropagation of negative weights for the reward signals for the various temperature and duration conditions used to generate a polynucleotide with a low fidelity.

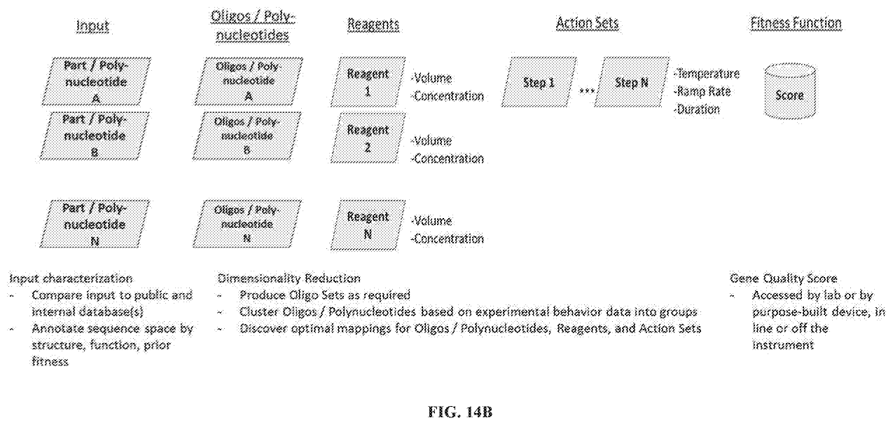

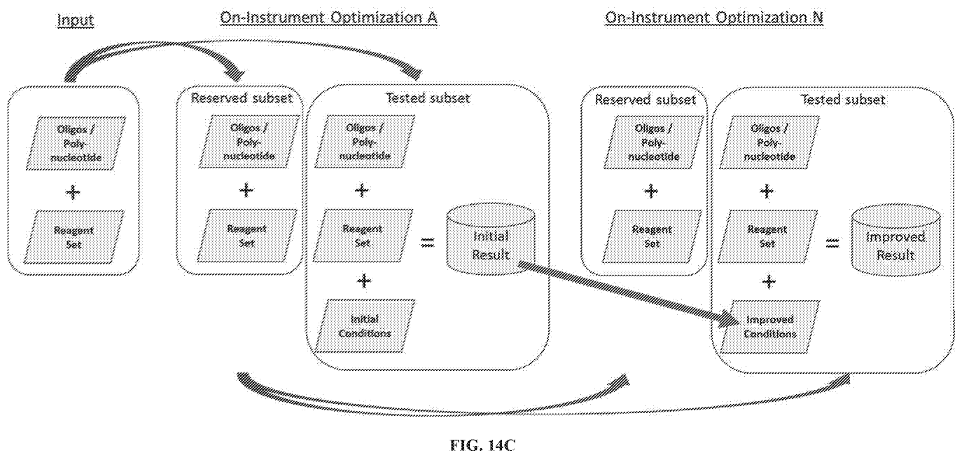

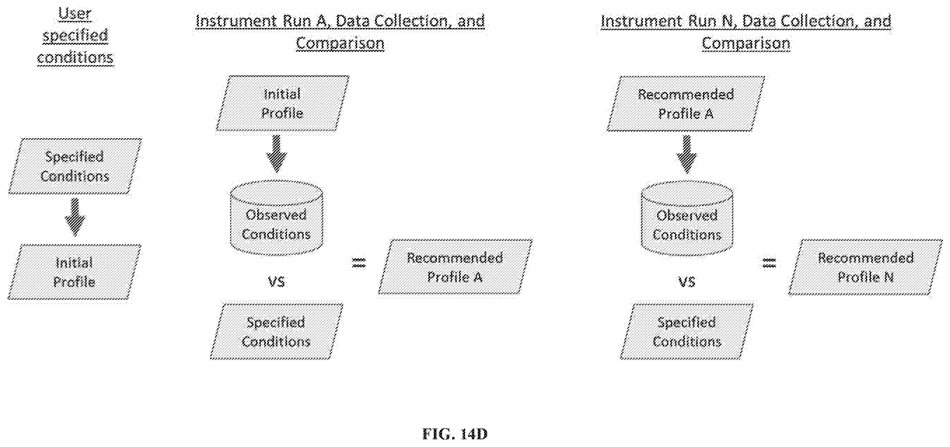

An example of a solution search space for the reinforcement learning process is shown in FIG. 14A. The reinforcement learning can optimize various solutions search spaces in generating a recommendation of a design of experiment for a polynucleotide synthesis of a polynucleotide of interest. In some embodiments, the solutions search spaces comprise a biopart (i.e. oligonucleotides), a reagent condition (i.e. volume or concentration of a reagent), an action set (i.e. temperature, ramp rate of temperature, duration of a temperature), and a fitness function (i.e. a polynucleotide quality score from experimental results). In some embodiments, the reagents comprise at least one of nucleases, polymerases, ligases, nucleic acid molecules, buffers, and salts. In some embodiments, reagents comprise exonuclease, DNA polymerase, DNA ligase, and nucleic acid molecules. In some embodiments, dimensionality reduction can be performed to reduce computational cost and time. In some embodiments, dimensionality reduction can be performed to cluster oligonucleotides having similar behaviors into a group based on experimental results. In some embodiments, dimensionality reduction can be performed to discover optimal mapping for reagent and action sets based on the oligonucleotides sets. In some embodiments, dimensionality reduction can be performed to generate the oligonucleotides set required for an experimental design, as shown in FIG. 14B.

The device, methods, and systems herein can provide a clearinghouse for polynucleotide synthesis recipe information. In some embodiments, the disclosure herein comprises a cloud hosted database of polynucleotide synthesis recipe execution and optimization information. In some embodiments, the reinforcement learning algorithm is able to capture knowledge of synthetic biologists and those skilled in the art and automate the adjustments those skilled in the art may make to improve the fidelity of synthesized polynucleotide. In some embodiments, the disclosure herein allows a user to search and see what oligonucleotides or reagents and/or under what conditions the oligonucleotides or reagents have been used by other users. In some embodiments, the disclosure herein provides a design of experiment framework for polynucleotide synthesis. In some embodiments, the design of experiment framework can recommend action sets of best practices based on user inputs and constraints. As the optimization engine builds knowledge, recommendations may be based on prior experimental learning information. In some embodiments, the disclosure herein provides a genetic algorithm for discovery.

FIG. 14B shows an exemplary embodiment of a solution search space for the reinforcement learning process. The reinforcement learning process may take inputs from a user, or an AI system, or a combination thereof and design an experiment for a set of oligonucleotides and reagents having an action set from the solution search space of the AI system. After the set of oligonucleotides and reagents are prepared as recommended in the experiment design and loaded into the device provided herein, the device can apply the recommended action sets to assemble the target polynucleotides. The assembled target polynucleotides can be analyzed and compared to the target polynucleotide to generate a gene quality score. The gene quality score along with the inputs, set of oligonucleotides and reagents, and action sets can be provided to a fitness function to refine the solution search space of the AI system. In some embodiments, the inputs comprise at least one of a biopart, or a polynucleotide, or a combination thereof. In some embodiments, the inputs comprise oligonucleotides. In some embodiments, the biopart comprises at least one of a peptide sequence, a binding peptide sequence, an antibody sequence, or a combination thereof. In some embodiments, the input is received from a user. In some embodiments, the process run manually based on input by a user. In some embodiments, the process runs automatically based on recommendations from the AI system without any user input. In some embodiments, the process runs on a device provided herein specifically designed to the process. In some embodiments, the process runs on an in-line device that interfaces with the thermocycler provided herein. In some embodiments, the process runs based on a combination of user input and AI system input. In some embodiments, the AI system performs input characterization. In some embodiments, the input characterization comprises the AI system comparing the input data to public databases and internal database based on past reaction runs. In some embodiments, the input characterization comprises annotating the sequence space by structure, function, and prior experimental data, if available. In some embodiments, the AI system determines if the input data is similar to the data in public bioparts or biologic information databases. In some embodiments, the AI system checks if the input data is similar to prior input that had an Action set resulting in a Quality Score. In some embodiments, the information from input characterization informs the experimental design process. In some embodiments, any bioparts not represented by pre-existing polynucleotides are designed as oligonucleotides. In some embodiments, action sets are developed to produce new polynucleotides from oligonucleotides and assemble polynucleotides into final construct. In some embodiments, the action sets are executed to assemble polynucleotides. In some embodiments, the quality scores are determined for each action set. In some embodiments, the quality scores are generated from analysis of gels, chromatography, or sequencing of the resulting assembled polynucleotides from the action set. In some embodiments, the process is performed in iterations. In some embodiments, the recommendation from the AI system for an input data improves with additional iteration. In some embodiments, the improvement is a higher quality score. In some embodiments, the improvement is reduced dimensionality.

FIG. 14C shows an example of in situ experimental optimization. After a set of oligonucleotides and reagents can be loaded into the wells placed on the device described herein, the device can perform an on-instrument optimization process cycle comprising applying an action set of initial conditions of to a subset of the wells, referred herein as tested subset, to provide an initial result of the polynucleotide assembly. A subset of the wells, referred herein as reserved subset, may be reserved for a later run and remain unreacted by the initial action set. The initial result of the tested subset may be used to generate an improved action set having new improved conditions. The on-instrument optimization process cycle can be repeated at least one time with the new improved action set on a new subset of wells. In some instances, the instrument optimization process cycle can be repeated at least 1, 2, 3, 4, 5, 6, 7, 8, 9, 10, 11, 12, 13, 14, 15, 16, 17, 18, 19, 20, 25, or 30 times. The device can apply the improved action set that has been optimized from the tested subset results to the reserved subset to generate improved results. The in situ experimental optimization provide an advantage of saving in time and resources by conducting multiple small runs from one loading of the oligonucleotides and reagents and by having to open the lid only once for multiple small runs. In some embodiments, in situ experimental optimization comprises a cascading process by which a subset of reaction wells are reserved while another subset is tested with an initial set of conditions such that improved conditions can be derived and the process repeated. In some embodiments, all wells of an assay plate are loaded with identical oligonucleotides and reagents, and a subset of wells is run with varying conditions. In some embodiments, the results from the reactions of the subset of wells inform the next subset of wells. In some embodiments, the process is run until all held reactions are consumed and more optimal conditions are determined. In some embodiments, the subset of wells is at most 75%, 70%, 60%, 50%, 40%, 30%, 20%, or 10% of the total wells. In some embodiments, the subset of wells is at least 75%, 70%, 60%, 50%, 40%, 30%, 20%, or 10% of the total wells. In some embodiments, the subset of wells is at least 6, 12, 24, or 48 wells. In some embodiments, the subset of wells is at most 6, 12, 24, or 48 wells. In some embodiments, the tested subset has more wells than the reserved subset. In some embodiments, the later tested subset has less wells than previously tested subset in the same run. In some embodiments, the tested subset has same number of wells as reserved subset. In some embodiments, the tested subset has same set of oligonucleotides reagents of wells as reserved subset. In some embodiments, all wells have the same set of oligonucleotides reagents of wells.

FIG. 14D shows an example of instrument profile optimization with data collected during operation. The devices, methods, and systems described herein can have a module for measuring and recording various conditions of individual wells in real-time or approximately real-time over the course of a run, and the recording can be compared with the specified conditions to adjust the current run or future runs and to provide recommendation for future action sets of conditions. A set of specified conditions in action sets is provided to a device, and the specified conditions are executed and measured by the device. The measured conditions, also referred herein as observed conditions, and specified conditions over the course of the run can be compared to generate a new set of recommended conditions, also referred herein as recommended profile. In some embodiments, the condition comprises temperature, temperature change rate (i.e. heating rate), or volume, or a combination thereof. The condition can be taken at a predetermined time interval. In some embodiments, the condition is taken at every 1, 2, 3, 4, 5, 6, 7, 8 ,9, 10, 15, 20, 25, 30, 35, 40, 45, 50, 55, or 60 seconds. In some embodiments, the device compares the measured and specified conditions during the run and adjusts the conditions to more closely match the specified conditions. In some embodiments, the comparison is used to tune the device to more closely match the specified conditions during the same run or in future runs. In some embodiments, the measured conditions over the course of the run are provided to a compensation algorithm to adjust the device, where the measured conditions vary over time. In some embodiments, the device compares the measured and specified conditions after the run. In some embodiments, the comparison between the measured and specified conditions occurs after the run. In some embodiments, the device provides the measured conditions and the specified conditions in a transferable format, and the comparison of the measured and specified conditions is performed on a different machine, including but not limited to a computer or a tablet. In some embodiments, the comparison comprises coefficient of variation (CV) of between the specified and measured conditions. In some embodiments, the comparison comprises percent deviation between the specified and measured conditions. In some embodiments, the comparison comprises differences between the specified and measured conditions. In some embodiments, this is repeated until the instrument matches the specified conditions as best as possible. In some embodiments, the recordings and comparison are correlated to the polynucleotide quality score. In some embodiments, a poor polynucleotide quality score may be correlated with a large variation between the measured and specified conditions. In some embodiments, a high polynucleotide quality score may be correlated with a small variation between the measured and specified conditions. In some embodiments, the comparisons of measured and specified conditions are used to modify the polynucleotide quality score. In some embodiments, the comparisons of measured and specified conditions are a factor in determining future recommendations.

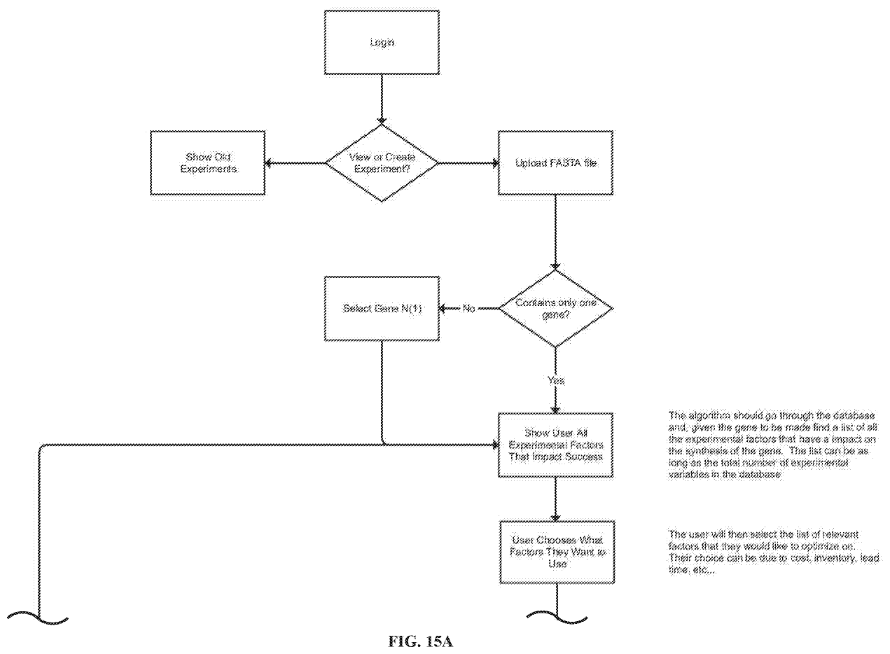

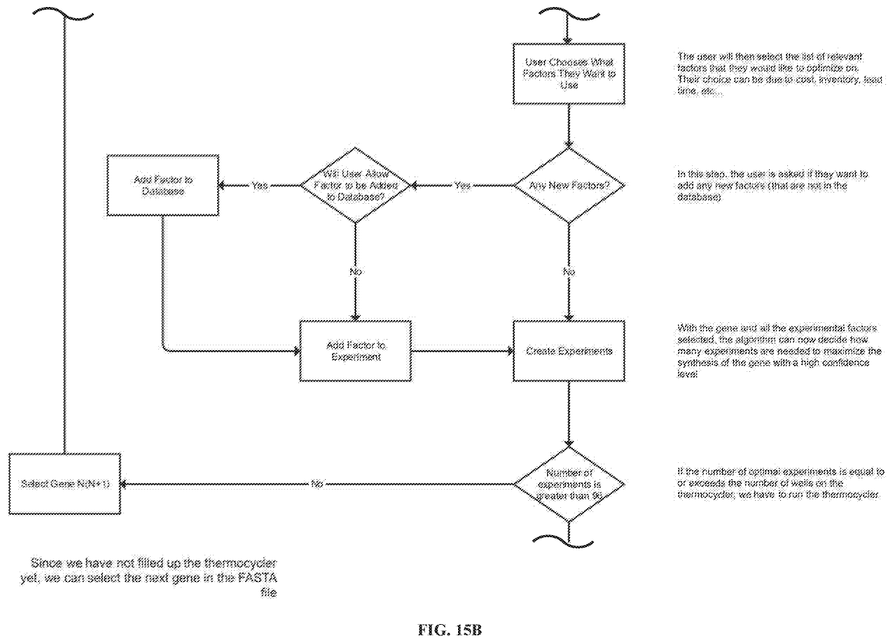

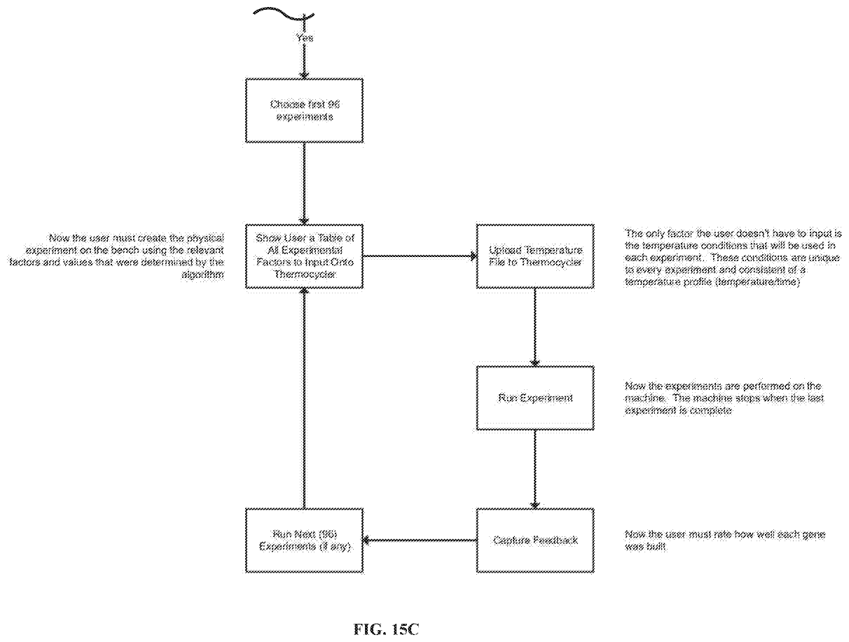

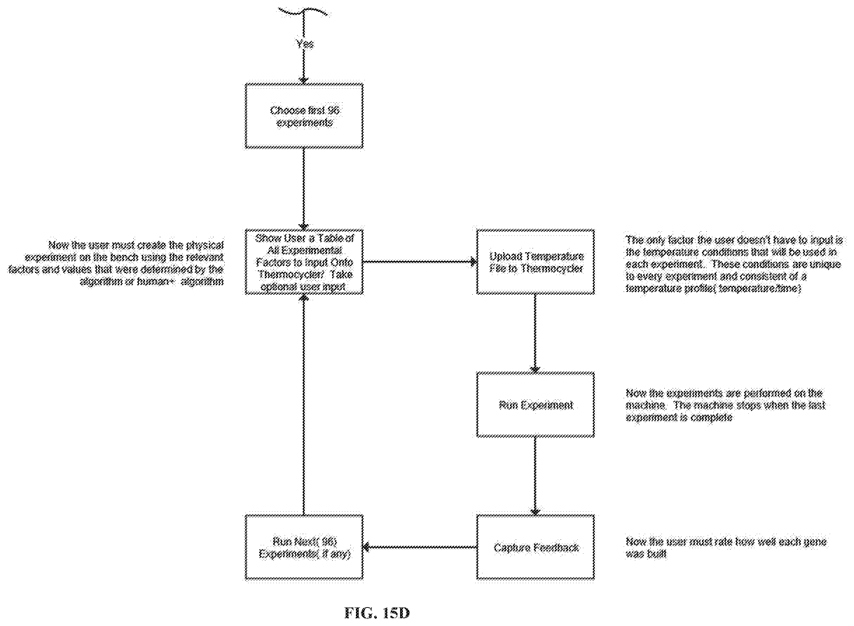

Exemplary flowcharts of the reinforcement learning in providing recommended experimental conditions for a polynucleotide synthesis in a web-based application are shown in FIGS. 15A-D. FIGS. 15A-C are sections of a continuing flowchart that flows down from FIG. 15A to FIG. 15B to FIG. 15C. In an exemplary embodiment, a user logs in to view or create an experiment. To create an experiment, the user may upload a FASTA file. In some embodiments, the FASTA file has more than one polynucleotide, and the user is prompted to select a polynucleotide of interest. In some embodiments, the FASTA file has only one polynucleotide. Then, the web-based application shows the user all experimental factors that impact the success or the fidelity of the synthesized polynucleotide. In some embodiments, the algorithm searches through the online database and generates a list of all the experimental factors that have an impact on the synthesis of the polynucleotide of interest. In some embodiments, the list can be as long as the total number of experimental variables in the database. The user can chooses which of the factor they want to use in the experiment. In some embodiments, the user can select the list of relevant factors that they would like to optimize on. In some embodiments, their choice can be due to cost, inventory, lead time, or other factors. Then, the user is asked if they want to add any new factors that are not in the online database to the experiment. If the user adds new factors, the user can be asked if they allow the new factors to be added to the database. Depending on the response of the user, the new factors may be added to the database. If the user adds new factors, the new factors are added to the experiment. Then, the experiment is created using the factors chosen by the user. With the polynucleotide and all the experimental factors selected, the algorithm decides how many experiments are needed to maximize the synthesis of the polynucleotide of interest with a high confidence level. If the number of optimal experiments is equal to or exceeds the number of wells on the thermocycler, the thermocycler may prepare to run the first experiment with the experiments that fit on the wells of the well plate on the thermocycler. If the number of optimal experiments is less than the number of wells on the thermocycler, the next polynucleotide of interest can be analyzed by the algorithm and added to the same run. Then, the user may create the physical experiment on the bench using the relevant factors and values determined by the algorithm. The web-based application can show the user a table of all experimental factors to prepare and place into the thermocycler with individually controlled chambers. The web-based application provides the temperature conditions for each experiment to the thermocycler with individually controlled chambers. In some embodiments, the conditions are unique to the individual experiment and consistent of a temperature profile (temperature/time). Then, the experiments are performed on the thermocycler, which stops when the last experiment is complete. After the polynucleotide synthesis is complete, the user rates the quality of the synthesized polynucleotide from each chamber. The feedback from the user is captured by the reinforcement learning algorithm and may be used in future searches.

FIG. 15D provides an alternative to FIG. 15C of a continuing flowchart that flows down from FIG. 15A to FIG. 15B to FIG. 15D. In an exemplary embodiment, after the algorithm decides how many experiments are needed to maximize the synthesis of the polynucleotide of interest with a high confidence level, the thermocycler may prepare to run the first experiment with the experiments that fit on the wells of the well plate on the thermocycler. In some embodiments, as shown in FIG. 15D, the device shows the user a table of all experimental factors to input onto the thermocycler and takes optional user input. In some embodiments, the user creates the physical experiment on the bench using the relevant factors and values that were determined by the algorithm or by a combination of human and the algorithm. In some embodiments, the temperature file is uploaded to the device comprising a thermocycler. In some embodiments, a factor that the user does not have to input is the temperature conditions that are used in each experiment run. These conditions are unique to every experiment and consistent of a temperature profile (i.e. temperature over time). The device runs the experiments to assemble target polynucleotides and stops when the last experiment is complete. In some embodiments, the user rates the quality of the assembled polynucleotides and the device captures the user feedback. In some embodiments, the system captures the quality of the assembled polynucleotides by in-line or otherwise connected device or computer system or database. In some embodiments, the next run is prepared and run.

A person of ordinary skill in the art will recognize many variations based on the teaching described herein as shown in FIGS. 15A-D. The steps may be completed in a different order. Steps may be added or deleted. Some of the steps may comprise sub-steps. Many of the steps may be repeated as often as advantageous to the disclosure herein.

### User Interface

In some embodiments, a web-based interface controls the devices described herein. In some embodiments, the web-based interface comprises software to control the thermocycler from the web. In some embodiments, a user controls the software to control the thermocycler. In some embodiments, the web-based interface provides remote programming and monitoring of the device. In some embodiments, the web-based interface comprises a mobile application or a web application that controls one or more devices. In some embodiments, the web-based interface uses machine learning to improve the polynucleotide synthesis process over time with additional runs. In some embodiments, the web-based interface provides for sharing of protocols amongst users and devices. In some embodiments, the web-based interface comprises tools to create a temperature profile for an individual well that is distinct from a temperature profile for another well in the same well plate. In some embodiments, the web-based interface allows for automation of protocol selection and/or robotic operation. In some embodiments, the web-based interface is an application programming interface (API). In some embodiments, the API allows for easy integration with laboratory information management system and automation.

In some embodiments, the devices, methods, and systems provided herein comprise a central database that consolidates all workflows, process variables and performance metrics from users. In some embodiments, the devices, methods, and systems provided herein use machine learning and artificial intelligence to find trends in the data provided by the users.

In some embodiments, the temperature protocols and the input process data are stored in a centralized database. In some embodiments, the centralized database has temperature protocols and the input process data are from multiple users. In some embodiments, the centralized database facilitates sharing of data by one user to another user. The sharing of data facilitates scientific collaboration by sharing of experimental protocols and collaborative optimization of the protocols using PCR (DNA assembly, cloning, library preparation, etc.).

In some embodiments, the input process data are provided to the devices, methods, and systems for polynucleotide synthesis. In some embodiments, the input process data comprises oligonucleotide design, primer design, components, or reagents. In some embodiments, the oligonucleotides are designed by (digitally) breaking up the target polynucleotide into short segments of single stranded oligonucleotides at various points. The points at which they are “cut” is critical to DNA assembly. In some embodiments, the primers are designed such that once the polynucleotide has been assembled from the oligonucleotides, subsequent PCR can be performed. In some embodiments, the components and reagents are provided in polynucleotide assembly kits. In some embodiments, the components and reagents comprise buffers, polymerase and dNTP's. In some embodiments, the components and reagents further comprise additives such as magnesium, DMSO, EDTA.

In some embodiments, the devices, methods, and systems for polynucleotide synthesis provide an output data. In some embodiments, quantitative output metrics post-assembly comprise sequences and concentrations of the polynucleotide product. In some embodiments, the synthesis result, including but not limited to the polynucleotide quality score, quality, and accuracy, is combined with the input process data and uploaded to the online database. In some embodiments, the systems and methods provided herein interfaces with a LIMS system to save the input process data and output data. In some embodiments, the systems and methods provided herein uses a web interface to save the input process data and output data. In some embodiments, the devices, systems, and methods provided herein use AI to find trends in the data and to make process recommendations to the user.

### Polynucleotide Synthesis Workflow

The disclosure herein may improve upon the existing system by allowing the synthesis of polynucleotides or any other thermally assembled macromolecule in a high throughput approach. This approach can be combined with optimized process conditions that are specially prepared by machine learning algorithms that learn from an online database. The device is “high throughput,” because it can perform many different runs with different temperature conditions and reaction mixture conditions in parallel, which can save the time as compared to serial runs. The thermocycler device is “smart,” because it interacts with the user who enters the process input conditions for the polymeric molecule of interest for synthesis.

In various embodiments disclosed herein, the device is capable of controlling independently the temperature condition of individual reaction chambers. In some embodiments, the device has an externally mounted touchscreen display, camera and microphone, or a combination thereof that are used to interface with the user. When a user decides to begin an experimental run, they can input their experimental factors in a number of ways, including but not limited to manually via touchscreen, voice commands, by scanning reagent barcodes in front of the camera or by presenting hand written notes, product labels, etc. The optical character recognition system in the device can take this unstructured data and turn it into a set of structured input variables. Nonlimiting examples of these input variables include oligonucleotides used, polynucleotides to be made, reagents, concentrations, volumes etc.

Once the run input variable process is complete, the device can relay all the data up to the online database where a unique series of machine learning algorithms will combine the custom input factors for this experiment with the complete archive of anonymous input variables of past experiments from other users operating other devices and use past run quality data to develop a set of optimized experimental conditions for this users particular process. These optimized process conditions will be sent back down to the device and applied to each well in the device.

Once the optimized process are uploaded to the device and the user loads a 96 well plate containing the starting solutions for their experimental run, the run sequence can begin. The microprocessor on the device begins this process by sending a signal to the baseplate motor which starts to rotate a baseplate rotating actuator 7, 8 in FIG. 1. This rotating actuator is in intimate contact with a fixed baseplate 13 as shown in FIG. 1. By performing this action, the entire rotating baseplate assembly can rotate from the load position to the run position as shown in FIG. 7. As this happens, the well plate moves underneath the heated lid 3 as shown in FIG. 1 and it is substituted with the well plate cover 17 as shown in FIG. 3 which cosmetically fits inside the void left without the well plate. Once the baseplate is finished rotating, the microprocessor signals the lid actuator motor 9 as shown in FIG. 1 to clamp down upon the core assembly and well plate. Additionally, when in the “run” position, the power coupling assembly 15, 16 as shown in FIG. 3 is engaged so that up to 80 A of current can be sent from the power supply 11 to the core assembly to power the high current thermoelectric modules.

The core assembly of the devices and systems disclosed herein allows for individual thermal control of individual chambers. The device provides precise temperature control (i.e. about 0.05° C.) and fast ramp rates (up to 5° C./s) of individual chambers. In some embodiments, the device is capable of providing temperature control of at least about 0.01° C., 0.02° C., 0.03° C., 0.04° C., 0.05° C., 0.06° C., 0.07° C., 0.08° C., 0.09° C., 0.1° C., 0.2° C., 0.3° C., 0.4° C., or 0.5° C. In some embodiments, the device is capable of providing temperature control to at most about 0.01° C., 0.02° C., 0.03° C., 0.04° C., 0.05° C., 0.06° C., 0.07° C., 0.08° C., 0.09° C., 0.1° C., 0.2° C., 0.3° C., 0.4° C., or 0.5° C. In some embodiments, the device is capable of providing temperature control of about 0.01° C., 0.02° C., 0.03° C., 0.04° C., 0.05° C., 0.06° C., 0.07° C., 0.08° C., 0.09° C., 0.1° C., 0.2° C., 0.3° C., 0.4° C., or 0.5° C. In some embodiments, the device is capable of temperature accuracy of at least about 0.01° C., 0.02° C., 0.03° C., 0.04° C., 0.05° C., 0.06° C., 0.07° C., 0.08° C., 0.09° C., 0.1° C., 0.2° C., 0.3° C., 0.4° C., or 0.5° C. In some embodiments, the device is capable of temperature accuracy of about 0.1° C. In some embodiments, the device is capable of temperature accuracy of about 0.05° C. In some embodiments, the temperature ramp rate is the heating rate. In some embodiments, the temperature ramp rate is the cooling rate. In some embodiments, the temperature ramp rate is for an individual chamber. In some embodiments, the device is capable of providing temperatures ramp rates of at least about 1° C./s, 2° C./s, 3° C./s, 4° C./s, 5° C./s, 6° C./s, 7° C./s, 8° C./s, 9° C./s, or 10° C./s. In some embodiments, the device is capable of providing temperatures ramp rates of about 5° C./s. In some embodiments, the device is capable of providing temperatures ramp rates of about 4° C./s. In some embodiments, the device is capable of temperature difference between neighboring wells of at least about 10° C., 20° C., 30° C., 40° C., 50° C., 60° C., 70° C., 80° C., 90° C., 100° C., 110° C., 120° C., or 130° C. In some embodiments, the device is capable of temperature difference between neighboring wells of no more than about 50° C., 60° C., 70° C., 80° C., 90° C., 100° C., 110° C., 120° C., 130° C., 140° C., or 150° C. In some embodiments, the device is capable of temperature difference between neighboring wells is about 90° C., 91° C., 92° C., 93° C., 94° C., 95° C., 96° C., 97° C., 98° C., 99° C., or 100° C. In some embodiments, the device is capable of temperature difference between neighboring wells is about 98° C. In some embodiments, the device is capable of temperature difference between neighboring wells is about 100° C. In some embodiments, the device has a temperature range of about 0° C.-100° C., 0° C.-95° C., 0° C.-90° C., −10° C.-100° C., or −20° C.-100° C.

The devices, methods, and systems provided herein use a temperature protocol to control the temperature setting on an individual well. In some embodiments, the temperature protocol is a time-dependent temperature setting. In some embodiments, the temperature protocol is a sequence of temperatures and temperature ramps over time, where each temperatures and temperature ramps are set for predetermined time frames. In some embodiments, the temperature protocol is chosen by the user. In some embodiments, the temperature protocol is automated. In some embodiments, the temperature protocol chosen based on the target polynucleotide. In some embodiments, the device uses a temperature protocol of an individual well that is independent of a temperature protocol for another well in the same well plate.

Many thermal cyclers currently on the market are manually actuated. This means that the user has to open and close the heated lid manually. The few automated solutions that are available on the market place the lid onto the well plate in one of two ways. These include placing the well plate into position and have a linear actuator move the well plate in one axis underneath the heated lid and then actuating a second linear actuator in another axis to “press” the heated lid onto the plate (U.S. Pat. No. 6,197,572). The other design “rotates” the lid into position above the well plate (U.S. Pat. No. 9,446,407 B2). The device listed in this disclosure operated by rotating the entire “core” assembly listed above underneath the heated lid and then actuating the heated lid so that it presses into the well plate. This is done by actuating the baseplate motor 7 which directly drives the baseplate rotating actuator 8 depicted in FIG. 1.

The thermal cycler device disclosed herein runs at 80 A of power when every reaction chamber is heated. Since the core of the device is rotating, routing moving power cables may be more difficult. To solve this problem, a custom, spring loaded power coupling device was designed that is a larger version of what are known in the electronics industry as “pogo pins”. This can be seen in FIG. 4.

When the core mechanism is rotated, the signal wires may rotate with it. To accomplish this, a “twisting” wire route was designed. This can be viewed when in the load position in FIG. 7.

The device serves as a way for the user to enter experimental factors via touchscreen, voice, optical character recognition or barcode.

The process factors and the results of the process run are fed back to the online database and are anonymously grouped with the global user base who have adopted the device. This globally grouped data is used optimize the runs for all users of the device.

The method for assembling the well sleeves is unique in that the well sleeve has an external thread that is screwed into a well housing with 96 internal threads. Each sleeve is then torqued down with a special slotted tool until the bottom face contacts the top surface of the thermoelectric device with the right pressure.

The device herein may be manufactured by a variety of methods. In an exemplary approach, the core assembly portion of the invention is made first by:
1. Soldering the 96 separate thermoelectric modules onto the TEC PCB.
2. This is then installed onto the base part comprising the core assembly. On this part, there are 4 separate positioning dowel pins that serve to align the rest of the core assembly “stack”.
3. Underneath this base part, the heat sink is secured.
4. Then, the TEC PCB is placed on top of the base part using the (4) corner index pins.
5. On top of the PCB, the threaded well baseplate is aligned and secured with fasteners.
6. Each well sleeve is then installed into the threaded well baseplate with a special slot tool and tightened to the proper torque and secured with Loc-Tite or similar thread adhesive
7. Next, the thermistor PCB is placed on top of the baseplate and aligned with the index pins
8. The thermistors are placed proximate to each well and bonded into place with a thermally conductive adhesive.
9. Once all the thermistors are bonded onto each well, their leads are soldered onto the contacts on the thermistor PCB.
10. On top of the thermistor PCB goes an electrically insulative gap pad that protects the exposed thermistor leadwires. This is also aligned with the corner index pins.
11. Finally, the core assembly cover is placed on top of the gap pad and secured.

The core assembly herein may be installed to the rotating baseplate by various methods. In an exemplary method, installation of core assembly with the rotating baseplate comprises:
1. The rotating baseplate is installed with the baseplate motor and rotating actuator.
2. The core assembly is installed into place.
3. The moving portion of the power coupling assembly is installed onto the baseplate.
4. The wires are routed.

In various embodiments disclosed herein, the user may use the device or the system herein on a laboratory bench. The user can either enter in a polynucleotide or a macromolecule of interest for synthesis via touchscreen or verbal instructions. Then, the system herein may prompt the user to choose any or all of the experimental factors determined by the machining learning algorithms in the online database. The system can interact with the user in providing information and prompting for input via camera, microphone or touchscreen display, or a combination thereof. Once all experimental factors have been decided by the user based on the recommendation by the online database, the user may initiate the experimental run. Based upon the experimental factors decided before the run, the instrument may retrieve the best set of process conditions based upon the machine learning algorithms in the online database. The system may run the individual process in the corresponding individual chamber.

Once the run is complete, the user may remove the well plate from the instrument and analyze the synthesized polynucleotides or macromolecules. The user may provide an objective quality metric associated of the synthesized polynucleotides or macromolecules synthesized in each chamber with its associated process condition. In some embodiments, for synthesized DNA, the quality metric can be based upon an agarose gel, sequencing, cell count, or other commonly used methods in the field. The device may prompt the user to upload the quality metric to the online database, where it may be pooled with other experimental factors and results.

The various embodiments disclosed herein is not limited to produce synthetic nucleic acid molecule. It is believed that the idea of using a laboratory instrument as an interactive device is novel and speech recognition and/or optical character recognition can be applied to any number of existing laboratory instruments including pipettes, liquid handling robots, plate cranes, plate sealers etc. The idea of optimizing experimental factors and/or process conditions may be applied to a multitude of instruments if such an instrument may interface with an online database. Further, the thermal portion of the device disclosed herein can be utilized in a high throughput device for solution synthesis, enzymology, proteolysis, or other similar approaches that can benefit from independent temperature control of individual reaction chambers.

The devices, methods, and systems disclosed herein provide an interactive, high throughput thermal processing system. The disclosures herein provide a system for recording experimental details via voice, barcode and optical character recognition. Disclosed herein is a system for providing a user-directed experimental recommendation based upon little experimental details of the desired end product. Disclosed herein is a pool of web-based user data that can be accessed by special machine learning algorithms to provide optimal process conditions.

The disclosures provided herein address various shortcomings in polynucleotide synthesis and work flow. For example, existing thermocyclers are not able to run 96 simultaneous process conditions with individual temperature conditions for individual chambers. In some instances, existing thermocyclers (and other lab equipment) are unable to gather experimental details from the user without direct input. In some cases, existing laboratory notebooks transcribe without interacting with an online database that contains pools of structured data to decide how the user provided unstructured data should be classified. In some instances, there are no centralized databases that contain global process data across an anonymous user set.

An exemplary work flow for polynucleotide synthesis comprises:
1. The user uploads the polynucleotide of interest to synthesize to the online database.
2. The AI (artificial intelligence) engine in the online database takes the polynucleotide and recommends a set of key factors that may serve as the ingredients (i.e. oligonucleotides, reagents, etc.) and/or process (temperature, duration, ramp) conditions necessary to make the polynucleotide. In other DOE tools, the user must select a set of factors as well as their values (if categorical) or range (if numeric). The AI disclosed herein, including but not limited to machine learning and reinforcement learning, automates this action of selection of these factors along with their values and/or range. Therefore, after entering the polynucleotide, the next thing the user sees is a list of these recommendations.
3. In this “factor preview” window, the AI may have the choice of using the “AI recommended” factors (and range) or can manually override them. The user can also select a “probability of success” where increasing the probability leads to an increase in the number of wells needed to create the polynucleotide since more permutations of the factors may be necessary.
4. Additionally in the “factor preview” window, the user can remove (what they consider to be) unnecessary factors and/or add factors.
5. If the user adds factors, the AI allows the user to choose (from the global pool of factors in the database) an existing factor along with the values and/or range OR define an entirely new factor (one not found in database) where the user can define the definition of the factor as well as its value and/or range.

The devices, methods, and systems disclosed herein provide an online database or a cloud database of polynucleotide synthesis recipe execution and optimization information that acts as a clearinghouse. In some embodiments, the input for the online database comprises a FASTA file having information of the target polynucleotide of interest and/or a list of oligonucleotides for the polynucleotide of interest. Alternatively, the list of oligonucleotides for the polynucleotide of interest may be auto-calculate from polynucleotide of interest. In some embodiments, the recipe for the thermocycler is provided by the machine learning algorithm in the online database based on the input. In some embodiments, the recipe for the thermocycler comprises types of reagents, volumes of reagents, concentration of reagents, temperature process parameters, target temperature, ramp rate, and duration. In some embodiments, the recipe optionally comprises vendor for vendor quality assessment. In some embodiments, the output of the online database comprises quality of polynucleotide produced. In some embodiments, the quality of the polynucleotide can be assessed by client directly or via tools such as Oxford Nanopore. In some embodiments, any number of types of reagents, volumes of reagents, concentration of reagents, temperature process parameters, target temperature, ramp rate, and duration can be provided in the recommendation of a recipe for the thermocycler.

As the search space of possible recipes is infinite, the possible recipes are too large combinatorially to evaluate even when breaking value ranges down into discrete steps. As such, machine learning in online database can be a powerful tool in evaluating the possible recipes to generate a recommendation of optimal recipes. In some embodiments, the machine learning algorithm comprises intelligent reduction of the search space of parameters based on analyzing historical experiments and the resulting quality of the produced polynucleotides. This approach merges traditional Design of Experiments (DoE) with machine learning-based optimization of parameters. DoE provides information regarding selecting variable or step sizes to reduce the possible number of experiments. Machine learning may be used to auto-recommend recipes or parts of recipes that have been optimal in the past for similar polynucleotide fragments and bioparts (i.e. oligonucleotides). In some embodiments, if there is a partial match with previous experiment (reagent, volume/concentration, temperature, etc), the algorithm will do fuzzy/intelligent match to see if “similar” recipe has been executed in the past; and return the associated quality.

In some embodiments, reinforcement learning provides information on experiments performed with feedback for quality of synthesized products and facilitates determining optimal control strategy or recipe for high quality for given constraints. In some embodiments, the polynucleotide synthesis is represented as a recipe process having complex interactive components with nonlinear collective activities. In some embodiments, genetic algorithm can be used to generate random control recipes and reinforcement learning can be used to choose best rule. In some embodiments, an external state of these algorithms is modified by each action. In some embodiments, an action set comprises sequence of actions selected from an action list.

An exemplary approach of using online database for optimizing experimental conditions comprises various components. In some cases, a control/execution component comprises controls interactions with environment. In some cases, a reinforcement component distributes reward from environment to classifiers with optimal genetic quality output. In some cases, a discovery component exploits genetic algorithm to discover better recipes and improve existing ones according to fitness estimates. In some cases, slight tweaks to traditional reinforcement learning can be utilized, where instead of classification, numerical score output (genetic quality) is provided. This tweaked version of reinforcement learning may be a regression form of reinforcement learning.

The polynucleotide described herein can be of any of a variety of lengths. In some instances, the polynucleotide has a length of at least about 100 bases, 200 bases, 300 bases, 400 bases, 500 bases, 600 bases, 700 bases, 800 bases, 900 bases, 1 kilobase (kb), 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 200 kb, 300 kb, 400 kb, 500 kb, 600 kb, 700 kb, 800 kb, 900 kb, or 1 megabase (Mb). In some instances, the polynucleotide has a length of about 100 bases, 200 bases, 300 bases, 400 bases, 500 bases, 600 bases, 700 bases, 800 bases, 900 bases, 1 kb, 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 200 kb, 300 kb, 400 kb, 500 kb, 600 kb, 700 kb, 800 kb, 900 kb, or 1 Mb. In some instances, the polynucleotide has a length of at most about 900 Mb, 800 Mb, 700 Mb, 600 Mb, 500 Mb, 400 Mb, 300 Mb, 200 Mb, 100 Mb, 90 Mb, 80 Mb, 70 Mb, 60 Mb, 50 Mb, 40 Mb, 30 Mb, 20 Mb, 10 Mb, 9 Mb, 8 Mb, 7 Mb, 6 Mb, 5 Mb, 4 Mb, 3 Mb, 2 Mb, 1 Mb, 900 kb, 800 kb, 700 kb, 600 kb, 500 kb, 400 kb, 300 kb, 200 kb, 100 kb, 90 kb, 80 kb, 70 kb, 60 kb, 50 kb, 40 kb, 30 kb, 20 kb, 10 kb, 9 kb, 8 kb, 7 kb, 6 kb, 5 kb, 4 kb, 3 kb, 2 kb, or 1 kb.

The size of the oligonucleotides described herein may be any of a variety of lengths. In some cases, the oligonucleotides comprises nucleic acid molecules having a length of at least 2 bases, 5 bases, 10 bases, 15 bases, 20 bases, 25 bases, 30 bases, 35 bases, 40 bases, 45 bases, 50 bases, 75 bases, 100 bases, 125 bases, 150 bases, 175 bases, 200 bases, 225 bases, 250 bases, 275 bases, or 300 bases. In some cases, the oligonucleotides comprises nucleic acid molecules having a length of about 2 bases, 5 bases, 10 bases, 15 bases, 20 bases, 25 bases, 30 bases, 35 bases, 40 bases, 45 bases, 50 bases, 75 bases, 100 bases, 125 bases, 150 bases, 175 bases, 200 bases, 225 bases, 250 bases, 275 bases, or 300 bases. In some cases, the oligonucleotides comprises nucleic acid molecules having a length of at most about 300 bases, 275 bases, 250 bases, 225 bases, 200 bases, 175 bases, 150 bases, 125 bases, 100 bases, 75 bases, 50 bases, 45 bases, 40 bases, 35 bases, 30 bases, 25 bases, 20 bases, 15 bases, 10 bases, 5 bases, or 2 bases.

A synthesized polynucleotide with a high quality can have a high fidelity to the polynucleotide of interest. In some embodiments, a high fidelity refers to least 75%, at least 80%, at least 85%, at least 90%, at least 91%, at least 92%, at least 93%, at least 94%, at least 95%, at least 96%, at least 97%, at least 98%, or at least 99% match between the synthesized polynucleotide and the polynucleotide of interest. In some embodiments, a high fidelity refers to about 75%, 80%, 85%, 90%, 91%, 92%, 93%, 94%, 95%, 96%, 97%, 98%, or 99% match between the synthesized polynucleotide and the polynucleotide of interest. In some embodiments, a high fidelity refers to 100% match between the synthesized polynucleotide and the polynucleotide of interest.

TABLE 1
TABLE 1 provides a list of the various items in the
figures provided herein

| Item | Item Description | Drawing Description |
|---|---|---|
| 1 | Core Assembly | Complete Assembly |
| 2 | Rotating Baseplate | Complete Assembly |
| 3 | Heated Lid | Complete Assembly |
| 4 | Moving Power Cable Assembly | Complete Assembly |
| 5 | Fixed Power Cable Assembly | Complete Assembly |
| 6 | Power Coupling Assembly | Complete Assembly |
| 7 | Baseplate Motor | Complete Assembly |
| 8 | Baseplate Rotating Actuator | Complete Assembly |
| 9 | Lid Actuator | Complete Assembly |
| 10 | External Housing | Complete Assembly |
| 11 | DC Power Supply | Complete Assembly |
| 12 | Power Input Module | Complete Assembly |
| 13 | Fixed Baseplate | Complete Assembly |
| 35 | Slip Ring | Complete Assembly |
| 17 | Well Plate Cover | Core Assembly |
| 15/16 | Power Coupling Assembly | Core Assembly |
| 14 | Heat Exchanger | Core Assembly |
| 26 | Electrode Spring | Power Coupling |
| 27 | Fixed Electrode | Power Coupling |
| 28 | Moving Electrode | Power Coupling |
| 29 | Slotted Well Sleeve | Heated Sleeve Detail |
| 30 | Well Sleeve Thread | Heated Sleeve Detail |
| 31 | Thermoelectric Module | Heated Sleeve Detail |
| 32 | Thermistor | Heated Sleeve Detail |
| 30 | Threaded Well Baseplate | Exploded Core Assembly |
| 31 | Thermoelectric Module | Exploded Core Assembly |
| | (Mated to PCB) | |
| 32 | Thermistor | Exploded Core Assembly |
| 33 | Thermistor PCBA | Exploded Core Assembly |
| 34 | Cover Plate | Exploded Core Assembly |

### System

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

Referring to FIG. 4, in a particular embodiment, an application provision system comprises one or more databases 400 accessed by a relational database management system (RDBMS) 410. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 420 (such as Java servers, .NET servers, PHP servers, and the like) and one or more web servers 430 (such as Apache, IIS, GWS and the like). The web server(s) optionally expose one or more web services via app application programming interfaces (APIs) 440. Via a network, such as the Internet, the system provides browser-based and/or mobile native user interfaces.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable compiled applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of spotlight tour configuration information such as spotlight tour navigation steps, spotlight tour objects, spotlight tour shape properties, and spotlight tour controls data. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Definitions

The practice of some methods disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

The terms “about” and “approximately” mean within an acceptable error range for the particular value as determined by one of ordinary skill in the art, which will depend in part on how the value is measured or determined, such as the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about,” meaning within an acceptable error range for the particular value, should be assumed.

As used herein, the terms “polynucleotide”, “nucleic acid,” “oligonucleotide,” and “gene” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, isolated DNA of any sequence, cDNA, recombinant polynucleotides, branched polynucleotides, plasmids, vectors, isolated RNA of any sequence, messenger RNA (mRNA), transfer RNA (tRNA), ribosomal RNA (rRNA), short interfering RNA (siRNA), short-hairpin RNA (shRNA), micro-RNA (miRNA), ribozymes, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. Generally, oligonucleotides as used herein are shorter than polynucleotides.

The term “strand,” as used herein, refers to a nucleic acid made up of nucleotides covalently linked together by covalent bonds, e.g., phosphodiester bonds. In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, the “first” and “second” strands, the “coding” and “noncoding” strands, the “Watson” and “Crick” strands, or the “sense” and “antisense” strands. The assignment of a strand as being a top or bottom strand is arbitrary and does not imply any particular orientation, function or structure.

A polynucleotide may have a 5′ end and 3′ end, referring to the end-to-end chemical orientation of a single strand of polynucleotide or nucleic acid. In a single strand of linear DNA or RNA, the chemical convention of naming carbon atoms in the nucleotide sugar-ring means that there generally exists a 5′ end which frequently contains a phosphate group attached to the 5′ carbon and a 3′ end which typically is unmodified from the ribose —OH substituent (hydroxyl group). In some cases, a polynucleotide may have a —OH substituent or a hydroxyl group at a 5′ end and —P group or phosphate group at a 3′ end. A phosphate group attached to the 5′-end permits ligation of two nucleotides, e.g., the covalent binding of a 5′-phosphate to the 3′-hydroxyl group of another nucleotide, to form a phosphodiester bond. Removal of the 5′-phosphate may inhibit or prevent ligation. The 3′-hydroxyl group is also important as it is joined to the 5′-phosphate in ligation.

The term “primer,” as used herein, generally refers to an oligonucleotide, either natural or synthetic, that is capable, upon forming a duplex with a nucleic acid molecule template, of acting as a point of initiation of nucleic acid synthesis and being extended from its 3′ end along the template so that an extended duplex is formed. The sequence of nucleotides added during the extension reaction may be determined by the sequence of the template nucleic acid molecule. Usually primers are extended by a DNA polymerase. Sometimes primers are extended by a reverse transcriptase. Primers are generally of a length compatible with their use in synthesis of primer extension products, and usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

The terms “isolated” and “isolating,” with reference to a nucleic acid molecule or nucleic acid molecule complex generally refer to a preparation of the substance (e.g., nucleic acid molecule, nucleic acid molecule complex, extension products thereof) devoid of at least some of the other components that may also be present where the substance or a similar substance naturally occurs or is initially obtained from (e.g., a biological sample, a sample reaction volume, e.g., a synthesis reaction volume, etc). For example, an isolated substance may be prepared using a purification technique to enrich it from a source mixture. Enrichment can be measured on an absolute basis or in terms of a concentration, for example in terms of weight per volume of solution, molecules per volume of solution, or any other appropriate measure.

The term “gene synthesis,” as used herein, refers to polynucleotide synthesis or polynucleotide assembly. Polynucleotide synthesis refers to the process of covalently linking a nucleotide to another to another nucleotide, an oligonucleotide to another oligonucleotide, or a nucleotide to an oligonucleotide to generate a strand of nucleic acids, oligonucleotides, or polynucleotides.

As used herein, the terms “gene of interest” and “polynucleotide of interest” are used interchangeably. The terms mean that the sequence of the polynucleotide is known and chosen before synthesis or assembly of the polynucleotide product.

As used herein, the terms “well” and “chamber” are used interchangeably. A well refers to a container capable of holding reagents for the polynucleotide synthesis.

### EXAMPLES

### Example 1

### Exemplary Device for Polynucleotide Synthesis

Provided herein is an exemplary device and system suitable for polynucleotide synthesis with the following instrument specification (Table 2). An exemplary device and system suitable for polynucleotide synthesis is shown in FIG. 17. The device is designed to independently control each well with accuracy and simplicity. The device can independently control various settings of the 96 wells, including but not limited to temperature settings. The device can integrate lab automation using an automated lid and API. The device is designed to have a web-based software that provides an easy-to-use interface and traceability of the steps within any single well. The web-based software also provides enhanced collaboration among users, who can be local to the device or remote from the device.

TABLE 2
Device Specifications

| | |
|---|---|
| Block Format | 96-well microplate, 0.2 mL; |
| | 8-well strips |
| Max heating rate (well) | 3° C./s |
| Max cooling rate (well) | 2.5° C./s |
| Temperature accuracy | ±0.1° C. |
| Temperature range | 0-100° C. |
| Maximum temperature difference | 98° C. |
| (well to well) | |
| Dimensions (H × W × D) | 40 cm × 40 cm × 30 cm |
| Weight | 14 kg |
| Volume range | 10-100 μL |
| Display | 8-inch color TFT LCD |
| Power | 100-240 V, 50-60 Hz, max. 700 W |
| Data connectivity | Cloud or mobile via Ethernet, |
| | WiFi or USB |
| Protocol Storage | Unlimited with web app |
| Number of users | Unlimited with web app |
| Lid pressure | Up to 27 kg |
| Integration with automation | Via API |

### Example 2

### Exemplary Thermocylcling Device—Multitherm

Provided herein is an exemplary device for polynucleotide synthesis. The device comprises a 96 well thermocycler with individual well control. The device is automatable and is controlled via protocols stored on web-app.

### Example 3

### Datatherm

Provided herein is an exemplary system comprising a recommendation engine for PCR optimization and design of experiment (DOE). The recommendation engine provides recommendation on which reagents at what concentrations and volumes to use in the synthesis protocol.

### Example 4

### Exemplary Thermocylcling Device—Minitherm

Provided herein is an exemplary device for polynucleotide synthesis. The device comprises a miniature thermocycler that is capable of thermal control and monitoring of individual wells. The device is small enough to be used on a bench top, modular as to include additional channels, and has a low cost. The device is a modular device that can be powered over USB. The device can be controlled remotely or by USB.

### Example 5

### Exemplary System—Liquitherm/Datatherm

Provided herein is an exemplary system comprising a microfluidic liquid handling technology that is reconfigurable. The system comprises a liquid handling robot and has in-situ sensing capabilities to monitor conditions. The system can interface with a miniature thermocycler as described in Example 4. The system can be used to automatically monitor and control liquid handling workflows and variables to accommodate the desired biological product and application.

### Example 6

### Exemplary Device, Method, and System

Provided herein is an exemplary method comprising chip-based quality control methods. The method comprises UV/Vis, electrophoresis, and next generation sequencing methods. Also provided herein is an exemplary device that is integrated with the system as described in Example 5 (MiniTherm and LiquiTherm) to form a fully reconfigurable, closed loop workflow (liquid handling, thermocycling and feedback). Also provided herein is an exemplary system using a refined recommendation engine with additional process variables, protocols, and applications.

### Example 7

### Exemplary Web-Based Interface

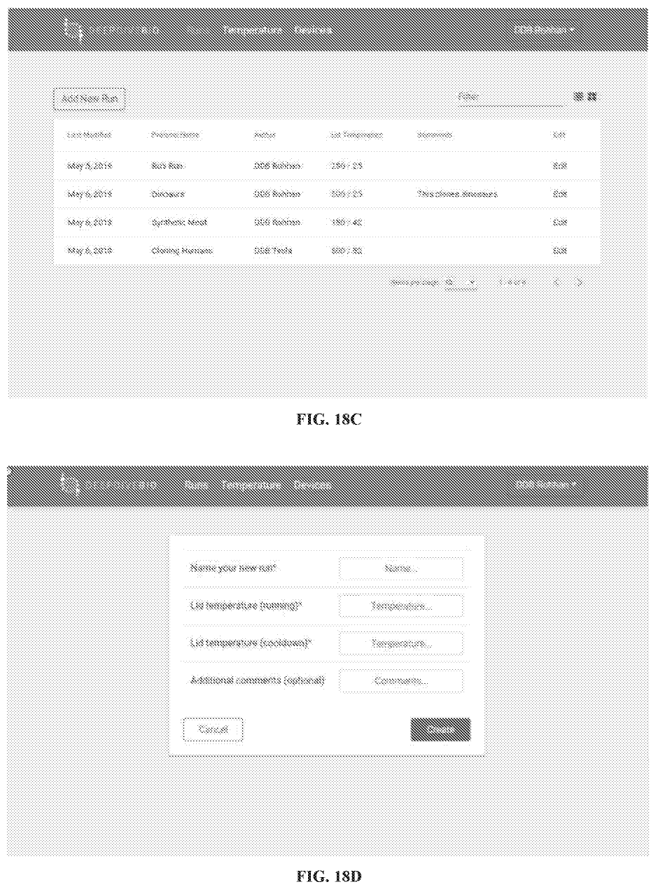

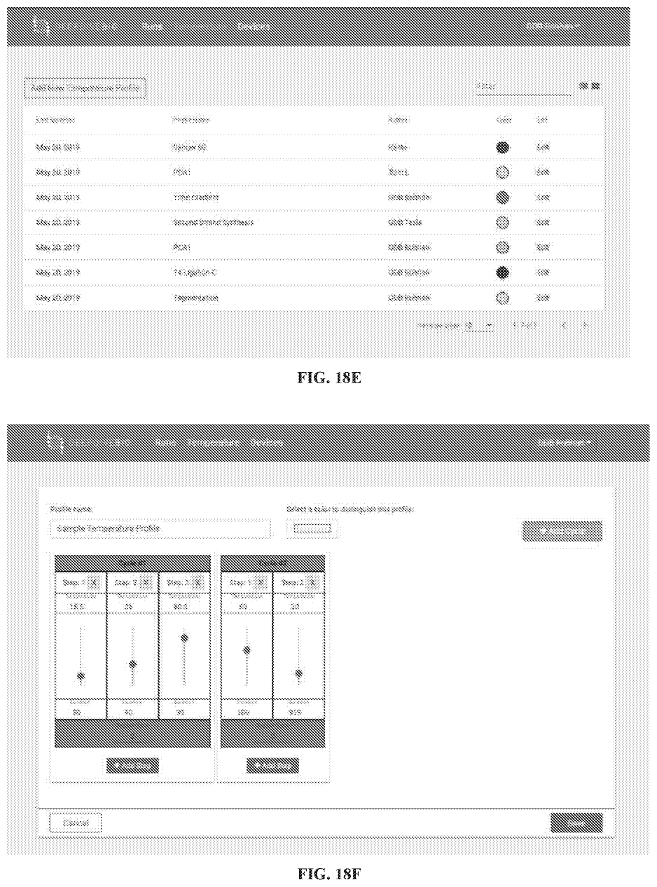

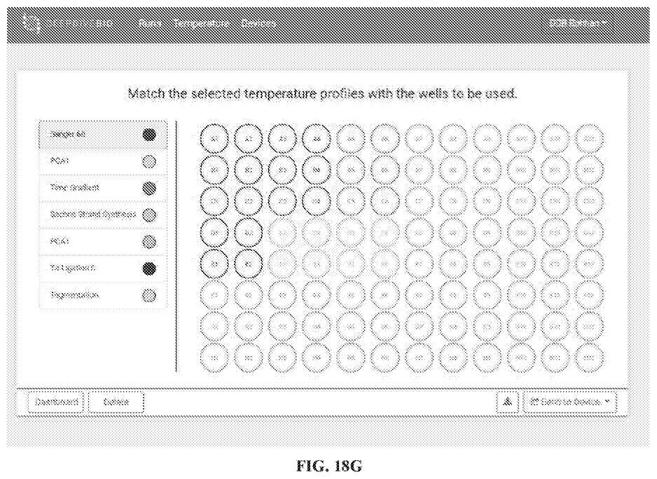

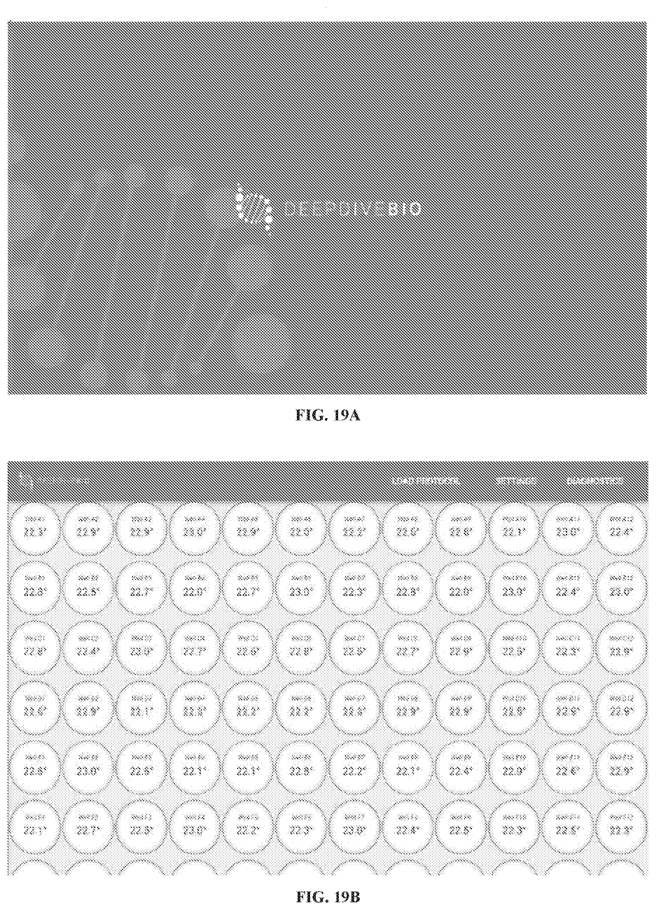

Provided herein is an exemplary web-based interface that is used to control the devices, systems, and methods provided herein. The interface can be categorized into 3 categories: 1) onboarding, 2) data viewing and sharing, and 3) setting up and running experiments. FIG. 18A shows the Login Screen where a user can sign up, log in, and read the Terms of Service Policy on this page. If the user is signing up for the first time, a verification code is sent to an email address provided by the user. After the user inputs their verification code, the user will be prompted to create their account. A user's “display name” is the name shown on the dashboard display and is the name linked to the data the user shares with their team. FIG. 18B shows the Invite Screen where a user can join an existing team or account. The user is prompted to enter the team's invite code after the user signs in. Having a shared workspace in the portal allows the user to define and share their work with the rest of the team without having to redefine each experiment every time. FIG. 18C shows the Runs Dashboard which shows a list of ‘runs’ protocols, and gives the user the option to add, edit, or re-run a protocol. A run is one complete run across a thermocycler. A run can be made up of several different experiments with unique temperature profiles for each well. FIG. 18D shows the Create a Run screen where the user can select ‘Runs’ from the main navigation menu to create a new experiment run. The user can add a name for their run and input the running and cooling lid temperatures. FIG. 18E shows the Temperature Profile Dashboard which lists all of the temperature profiles that the user or members on their team have created. The user can edit, add, or search for specific temperature profiles from this page. FIG. 18F shows the Create New Temperature Profile Page where the user can click on “Temperature” from the main navigation menu, and then click “Add New Temperature Profile”. Give your new profile a name, and select a color to distinguish this specific profile from others. The user can type in or use the slider to select the temperature desired for their experiment. The user can also select the duration for specific temperatures in your cycle. A temperature profile comprises ‘n’ cycles, and each cycle can have multiple temperature steps. Once the user completes their temperature profile, the user can click save. FIG. 18G shows the Apply Temperatures Page where after creating one or more temperature profiles, and adding a new run, the user is able to apply temperatures to specific wells in your new run. The intuitive interface allows the user to easily match a temperature profile to one of the wells in the thermocycler. The user can run multiple protocols in parallel with unique temperature settings for each well. This screen also allows the user to download or send experiment data to a device in their account (internet of things (IoT) enabled).

### Example 8

### Exemplary Web-Based Interface

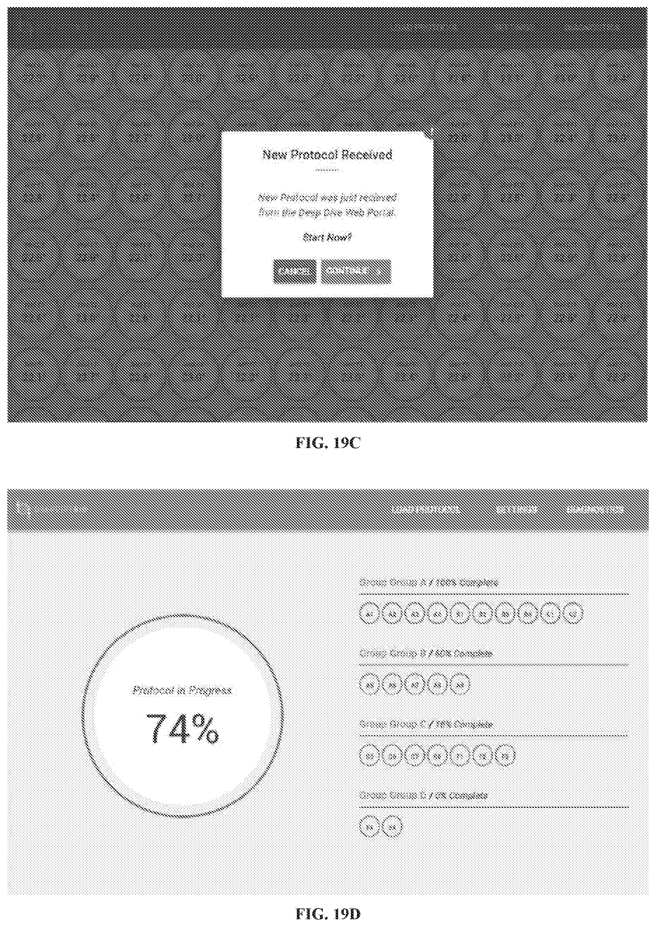

Provided herein is an exemplary web-based interface that is used to control the devices, systems, and methods provided herein. FIG. 19A shows the Splash screen, which is the first screen that pops up when the thermocycler is turned on, or when the thermocycler is idle. A user can tap anywhere on the screen to go into the portal. FIG. 19B shows the Wells screen, which displays the current temperatures for each well. The user can click on any well to adjust the temperature. FIG. 19C shows the Protocol Received screen. This modal pops up whenever a new protocol is received from an IoT-enabled device from the user's workspace. Protocols can also be uploaded through clicking on the “upload protocol” link. The thermocycler is capable of supporting both an online (IoT) and offline mode. In IOT mode, the user can send a protocol from anywhere to the device. It is also built in a way that is easy to integrate with LIMS systems. FIG. 19D shows the Protocol Progress screen, which shows progress as the protocol is run. FIG. 19E shows the Manual Control Screen that can be used alternatively or in combination with the online mode. In manual mode, the user can manually control the whole thermocycler. The user can adjust the lid temperature, move motors, and perform or direct various steps and actions.

### Example 9

### Exemplary Machine Learning Sequence

Provided herein is an exemplary machine learning sequence. A FASTA file is entered to the computing system by a user. An exemplary input is the nucleotide sequence for green fluorescent protein (GFP) as shown below:

>E17099.1 Aequorea victoria mRNA for green
fluorescent protein
ATGAGTAAAGGAGAAGAACTTTTCACTGGAGTTGTCCCAATTCTTGTTGAA
TTAGATGGTGATGTTAATGGGCACAAATTTTCTGTCAGTGGAGAGGGTGAA
GGTGATGCAACATACGGAAAACTTACCCTTAAATTTATTTGCACTACTGGA
AAACTACCTGTTCCATGGCCAACACTTGTCACTACTTTCTCTTATGGTGTT
CAATGCTTTTCAAGATACCCAGATCATATGAAACGGCATGACTTTTTCAAG
AGTGCCATGCCCGAAGGTTATGTACAGGAAAGAACTATATTTTTCAAAGAT
GACGGGAACTACAAGACACGTGCTGAAGTCAAGTTTGAAGGTGATACCCTT
GTTAATAGAATCGAGTTAAAAGGTATTGATTTTAAAGAAGATGGAAACATT
CTTGGACACAAATTGGAATACAACTATAACTCACACAATGTATACATCATG
GCAGACAAACAAAAGAATGGAATCAAAGTTAACTTCAAAATTAGACACAAC
ATTGAAGATGGAAGCGTTCAACTAGCAGACCATTATCAACAAAATACTCCA
ATTGGCGATGGCCCTGTCCTTTTACCAGACAACCATTACCTGTCCACACAA
TCTGCCCTTTCGAAAGATCCCAACGAAAAGAGAGACCACATGGTCCTTCTT
GAGTTTGTAACAGCTGCTGGGATTACACATGGCATGGATGAACTATACAAA
TAA

The computing system analyzes the input and compares the input to data from prior experiments. The computing system recommends factors and allows the user to select and/or add new factors. For example, the system recommends a temperature range for oligo annealing from 49° C. to 70° C. Then, the user manually changes the temperature range from the recommended range to a range from 48° C. to 72° C. The computing system recommends oligos for polynucleotide synthesis at a desired target length of 60 nucleotides. The user manually alters the desired target length to 63 nucleotides. The user informs the computing system the user input is complete. The following set of oligos for an oligo pool is generated by the computing system as shown in Table 3:

TABLE 3
Set of Oligonucleotides

| | |
|---|---|
| R1 | TCCAGTGAAAAGTTCTTCTCCTTTACTCAT |
| F1 | ATGAGTAAAGGAGAAGAACTTTTCACTGGAGTTGT |
| | CCCAATTCTTGTTGAATTAGATGGT |
| R2 | ACTGACAGAAAATTTGTGCCCATTAACATCACCAT |
| | CTAATTCAACAAGAATTGGGACAAC |
| F2 | GATGTTAATGGGCACAAATTTTCTGTCAGTGGAGA |
| | GGGTGAAGGTGATGCAACATACGGA |
| R3 | AGTAGTGCAAATAAATTTAAGGGTAAGTTTTCCGT |
| | ATGTTGCATCACCTTCACCCTCTCC |
| F3 | AAACTTACCCTTAAATTTATTTGCACTACTGGAAA |
| | ACTACCTGTTCCATGGCCAACACTT |
| R4 | GCATTGAACACCATAAGAGAAAGTAGTGACAAGTG |
| | TTGGCCATGGAACAGGTAGTTTTCC |
| F4 | GTCACTACTTTCTCTTATGGTGTTCAATGCTTTTC |
| | AAGATACCCAGATCATATGAAACGG |
| R5 | TTCGGGCATGGCACTCTTGAAAAAGTCATGCCGTT |
| | TCATATGATCTGGGTATCTTGAAAA |
| F5 | CATGACTTTTTCAAGAGTGCCATGCCCGAAGGTTA |
| | TGTACAGGAAAGAACTATATTTTTC |
| R6 | AGCACGTGTCTTGTAGTTCCCGTCATCTTTGAAAA |
| | ATATAGTTCTTTCCTGTACATAACC |
| F6 | AAAGATGACGGGAACTACAAGACACGTGCTGAAGT |
| | CAAGTTTGAAGGTGATACCCTTGTT |
| R7 | AAAATCAATACCTTTTAACTCGATTCTATTAACAA |
| | GGGTATCACCTTCAAACTTGACTTC |
| F7 | AATAGAATCGAGTTAAAAGGTATTGATTTTAAAGA |
| | AGATGGAAACATTCTTGGACACAAA |
| R8 | TACATTGTGTGAGTTATAGTTGTATTCCAATTTGT |
| | GTCCAAGAATGTTTCCATCTTCTTT |
| F8 | TTGGAATACAACTATAACTCACACAATGTATACAT |
| | CATGGCAGACAAACAAAAGAATGGA |
| R9 | GTTGTGTCTAATTTTGAAGTTAACTTTGATTCCAT |
| | TCTTTTGTTTGTCTGCCATGATGTA |
| F9 | ATCAAAGTTAACTTCAAAATTAGACACAACATTGA |
| | AGATGGAAGCGTTCAACTAGCAGAC |
| R10 | ATCGCCAATTGGAGTATTTTGTTGATAATGGTCTG |
| | CTAGTTGAACGCTTCCATCTTCAAT |
| F10 | CATTATCAACAAAATACTCCAATTGGCGATGGCCC |
| | TGTCCTTTTACCAGACAACCATTAC |
| R11 | ATCTTTCGAAAGGGCAGATTGTGTGGACAGGTAAT |
| | GGTTGTCTGGTAAAAGGACAGGGCC |
| F11 | CTGTCCACACAATCTGCCCTTTCGAAAGATCCCAA |
| | CGAAAAGAGAGACCACATGGTCCTT |
| R12 | TGTAATCCCAGCAGCTGTTACAAACTCAAGAAGGA |
| | CCATGTGGTCTCTCTTTTCGTTGGG |
| F13 | CTTGAGTTTGTAACAGCTGCTGGGATTACACATGG |
| | CATGGATGAACTATACAAATAAcca |
| F14 | tggTTATTTGTATAGTTCATCCATGCCATG |

The computing system further recommends reagents and reaction chamber conditions. The reagents comprise a standard Taq PCR reaction (NEB M0273): 10× Standard Taq Reaction Buffer at 1×, dNTPs at 200 μM, 10 μM Forward Primer at 0.2 μM (ATGAGTAAAGGAGAAGAACTTTTCACTGG), Reverse Primer at 0.2 μM (TTATTTGTATAGTTCATCCATGCCA), Oligo pool at 5 nM each oligo, Taq DNA Polymerase at 1.25 units/50 μl, and nuclease-free water for balance of the reaction.

The reaction reagents are mixed to produce at least 96×25 μl reactions and distributed across a 96 well-plate placed into the device described herein. The computing system recommends the time and temperature profiles, where the first step is at 95° C. for 2 minutes, then a cycle that repeats 30 times (95° C. for 30 cycles, an annealing temperature chosen from a range from 48° C. to 72° C. in 0.25° C. increments, and an extension of 1 min at 72° C.), followed by a final extension of 72° C. for 5 minutes. The thermocycler device is loaded with the mixed reagents and programmed with the conditions for each reaction. The reactions, each individual wells comprising an individual reaction, are run on the device.

A sample of the reaction is visualized by agarose gel electrophoresis with ethidium bromide and a DNA size marker loaded for reference. The images are provided to the computing system to calculate size and quality of the reactions.

The completed reactions are additionally individually cloned into a Topo cloning vector (ThermoFisher TOPO™ TA Cloning™ Kit for Sequencing, with One Shot™ TOP10 Chemically Competent E. coli TOPO™ TA Cloning™ Kit Catalog number: K457501) such that the resulting amplicons are cloned into the vector. The TOPO cloning reactions are transformed into TOP10 cells and plated on agarose plates with ampicillin (Teknova LB Agar Plates, Ampicillin-100, 100 mm #L1004). For each original reaction, 96 individual colonies are picked and miniprepped by growth in LB liquid media with ampicillin (Teknova LB Broth, Ampicillin-100, 1 L #L8105) and column purified (Qiagen #27104 QIAprep Spin Miniprep Kit), and Sanger DNA sequenced with M13 forward, and M13 reverse primers (ThermoFisher BigDye™ Terminator v3.1 Cycle Sequencing Kit Catalog number: 4337455 and analyzed on an ABI 3130xl genetic analyzer to generate sequence traces).

The sequence traces are provided to the computing system to calculate identity and error rates to be used to calculate a quality reward score for analysis by the AI computing system. The process is optionally repeated for further refinement.

The quality reward score, reagents, oligonucleotides, and reaction conditions are saved and stored by the computing system and are analyzed to generate recommendations for reagent, oligonucleotide, and/or reaction condition in future runs. The AI-generated recommendation help to reduce numbers of reactions as compared to a run of reactions without the AI-generated recommendation when a set of reagents, oligonucleotides, and reaction conditions that resulted in a low quality score in previous runs can be suggested to be removed from the run by the AI or removed by the user.

### Example 10

### Temperature Profile of Individual Wells on Well Plate Block

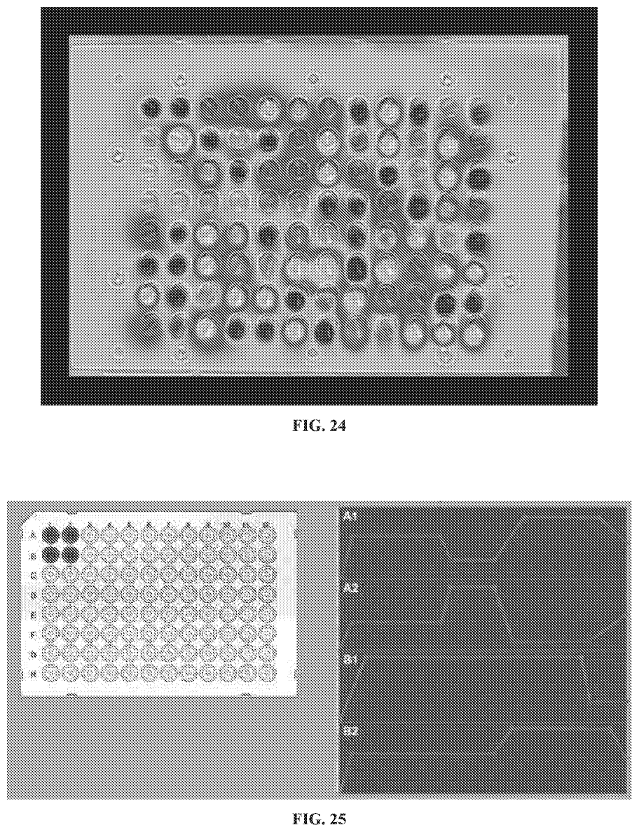

Provided herein is an exemplary well plate block of the devices, systems, and methods described herein. The well plate block can control the temperature of individual wells of a well plate placed on the well plate block. FIG. 24 shows a thermograph taken using a forward-looking infrared (FLIR) thermal imaging camera of a well plate on top of the well plate block. In the thermograph image, hot areas are represented by brighter colors and cooler areas are represented by darker areas. The individual wells have distinct circles with well-defined margins from their neighboring wells. This shows that individual wells are able to achieve different temperatures from its neighboring wells on the well plate block of the devices as described herein as indicated by differently colored circles corresponding to individual wells. FIG. 25 shows an example of four individual wells with distinct, different thermal profiles over a run. Each well has a distinct temperature profiles having various temperature ramps and temperatures at different times over a course of the run.

### Example 11

### Data Structure to Communicate with LIMS System

Provided herein in an exemplary method of control for the devices, systems, and methods described herein to communicate with the LIMS system. The method of control provides a way to control the device using external devices. The method of control uses a data structure that is of a standard format and is compatible for future updates and expansion of its capabilities. The method of control, also referred to as the Control Instructions, implements the specified conditions of the individual wells over time in seconds in JavaScript Object Notation (JSON). The method of control can use other file formats. The JSON object is provided to the device by at least one of direct transmission or remote transmission (e.g. over the air).

Over the Air: The device provides a way to connect to the Web Portal via an IoT messaging platform. A user is able to log into the online portal, select the device to be used, and send it a set of Control Instructions. The device is required to be connected to the internet. The device is required to be registered with an organization or a group that the user is a part of when the user uses the organization or group log-in information.

Stand Alone Solution: The device can be used without an internet connection and provides functionality to read the Control Instructions via a file on a USB Drive or other direct connection. The user navigates through menu options on the device to select and load the file.

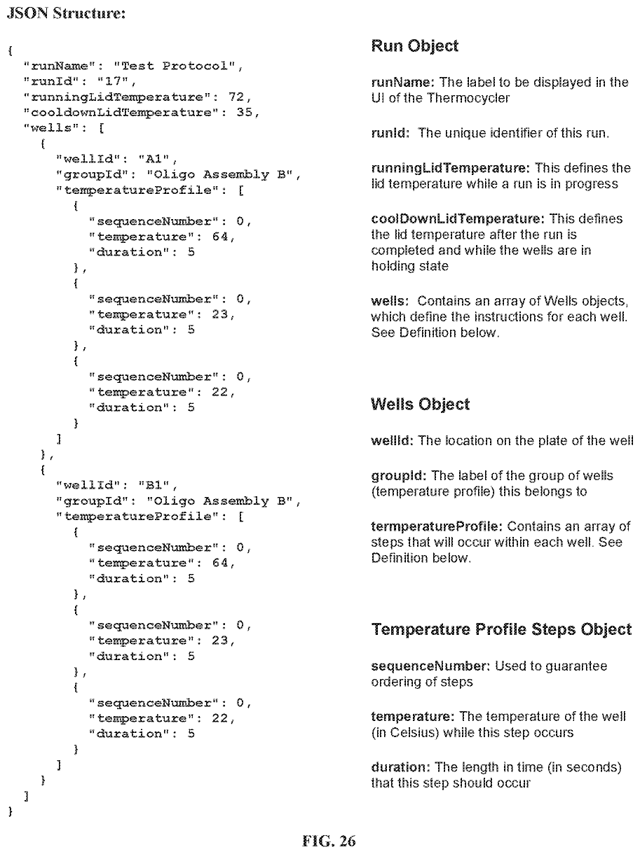

An exemplary JSON structure for the Control Instructions is shown in FIG. 26. The JSON structure comprises run object, wells object, and temperature profile steps object. The run object comprises data for a run name to provide a label to be displayed in the user interface of the device, a run identifier, a running lid temperature to define the lid temperature while the run is in progress, a cool down lid temperature to define the lid temperature after the run is completed and the wells in holding state, and wells to provide an array of wells object which define the instructions for each well. The wells object comprises data for a well ID to define the location of the well on the well plate, group ID to define the label of the groups of wells, and temperature profile to provide an array of steps that occurs within each individual well. The temperature profile steps object comprises data for sequence number that is used for ordering of steps, temperature to define the temperature of each well in Celsius during a specific step, and duration for the length of time in seconds during the specific step in a run.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A device for thermocycling for polynucleotide synthesis, comprising a plurality of reaction chambers, wherein a temperature setting of an individual reaction chamber can be controlled independent from a temperature setting of another individual reaction chamber.

2. The device of claim 1, wherein the device comprises a rotating baseplate, a motor, and a rotating actuator, wherein the plurality of reaction chambers is configured to rotate using the rotating baseplate, the motor, and the rotating actuator.

3. The device of claim 1, wherein the device comprises a microprocessor, thermoelectric modules, thermistors (temperature sensors), a heated lid, a heat sink fan, and a power coupling assembly to provide control of the temperature setting of the individual reaction chambers.

4. The device of claim 3, wherein the thermoelectric module comprises a thermoelectric cooler (TEC) board and a heat sink, wherein the TEC board comprises a plurality of thermal adhesive pads on a backing material.

5. The device of claim 4, wherein the plurality of thermal adhesive pads are spaced apart in similarly as the plurality of reaction chambers.

6. The device of claim 3, wherein the thermistor is in contact with an outer wall of the individual reaction chamber.

7. The device of claim 1, wherein the device comprises a touchscreen display, a camera, or a microphone, or a combination thereof for interfacing with a user.

8. The device of claim 1, wherein the device comprises an online database comprising an artificial intelligence, wherein the online database stores data from a plurality of polynucleotide synthesis reactions, wherein the data comprises reagent conditions, reaction chamber conditions, and quality scores of polynucleotide products.

9. The device of claim 8, wherein the online database connects to a web-based application, wherein the web-based application takes an input from a user and displays an output to a user.

10. The device of claim 1, wherein the plurality of reaction chambers comprises 96 individual reaction chambers.

11. The device of claim 1, wherein the temperature setting of the individual reaction chambers is controllable to at least 0.5° C.

12. The device of claim 11, wherein the temperature setting of the individual reaction chambers is controllable to about 0.05° C.

13. The device of claim 1, wherein the temperature setting of the individual reaction chambers has a temperature ramp at least 1° C./s.

14. The device of claim 13, wherein the temperature setting of the individual reaction chambers has a temperature ramp of about 5° C./s.

15. The device of claim 1, wherein the device interfaces with an online database comprising an artificial intelligence, wherein the artificial intelligence generates a report of a recommendation to synthesize a polynucleotide of interest having a high fidelity and wherein the recommendation comprises reagent conditions and reaction chamber conditions.

16. The device of claim 15, wherein the generating of the report of the recommendation by a computing system comprises:
(a) determining, by artificial intelligence, reagents and reaction chamber conditions to include in the recommendation based on the data of reagents and the data of reaction chamber conditions in the artificial intelligence; and
(b) determining, by artificial intelligence, a sequence of the connections of the data of reagents and the data of reaction chamber conditions that provides a polynucleotide quality score above a threshold score as the recommendation.

17. The device of claim 15, wherein the device interfaces with the online database by a touchscreen display, a camera, or a microphone, or a combination thereof.

18. The device of claim 17, wherein the touchscreen display, the camera, or the microphone, or the combination thereof transmits an input from a user to the online database and displays the report to the user.

19. The device of claim 18, wherein the user can modify the report using the touchscreen display, the camera, or the microphone, or the combination thereof before the report is executed by the device.

20. The device of claim 15, wherein the online database connects to a web-based application, wherein the web-based application takes an input from a user and displays an output to a user.

21. The device of claim 15, wherein the report is provided in a format compatible with the device.

22. The device of claim 15, wherein the report provides assignments of a reaction mixture and a reaction chamber condition to a reaction chamber of the thermocycling device.

23. The device of claim 15, further comprising providing the data of reaction chamber conditions to the thermocycling device; and controlling the reaction chamber conditions of an individual reaction chamber of the thermocycling device after the individual reaction chamber is filled with a corresponding reaction mixture based on the report.

24. The device of claim 23, comprising controlling reaction chamber conditions of a first subset of individual reaction chambers, wherein the first subset is less than the plurality of the reaction chambers, and generating an initial result.

25. The device of claim 24, comprising controlling reaction chamber conditions of a second subset of individual reaction chambers based on a second recommendation, wherein the second recommendation is generated using the initial result.

26. The device of claim 23, wherein the device comprises a module for measuring the reaction chamber conditions of an individual reaction chamber during a run.

27. The device of claim 25, the device adjusts the reaction chamber conditions of an individual reaction chamber during the run based on the measured reaction chamber conditions.

28. The device of claim 25, wherein the device adjusts the recommendation based on the measured reaction chamber conditions.

29. The device of claim 25, wherein the device provides a report of the measured reaction chamber conditions.

30. A computer-implemented method of generating a recommendation of a design of experiment for a polynucleotide synthesis comprising:
(a) obtaining data of a target molecule;
(b) applying, by a computing system, the data to a trained artificial intelligence, wherein training of the artificial intelligence comprises:
(i) assigning a connection between one of data of reagents and one of data of reaction chamber conditions or between one of data of reagents and another of data of reagents;
(ii) assigning a weight to the connection;
(iii) generating a reward signal from a polynucleotide quality score; and
(iv) updating the weight based on the reward signal;
(c) determining, by the computing system, a recommendation of a design of an experiment by the artificial intelligence, wherein determining comprises:
(i) determining reagents and reaction chamber conditions to include in the recommendation based on the data of reagents and the data of reaction chamber conditions in the artificial intelligence; and
(ii) determining a sequence of the connections of the data of reagents and the data of reaction chamber conditions that provides a polynucleotide quality score above a threshold score as the recommendation;
(d) generating, by the computing system, a report of the recommendation, wherein the recommendation comprises the data of reagents and the data of reaction chamber conditions from the sequence.

31. The method of claim 30, wherein the target molecule is a polynucleotide.

32. The method of claim 30, wherein the artificial intelligence comprises machine learning.

33. The method of claim 31, wherein the machine learning comprises reinforcement learning.

34. The method of claim 30, wherein the reinforcement learning comprises heuristic optimization, wherein the heuristic optimization reduces a number of the sequences.

35. The method of claim 30, further comprising applying, by the computing system, a dimensionality reduction prior to step (b).

36. The method of claim 35, wherein the dimensionality reduction comprises clustering of data of oligonucleotides into groups, wherein the groups are based on similar experimental behaviors of the oligonucleotides.

37. The method of claim 35, wherein the dimensionality reduction provides an improvement in mapping of sequences, wherein the improvement is characterized by improvement in polynucleotide quality score, computing time, or computing cost.

38. The method of claim 30, wherein the data of reagents comprises data of a reagent identification, a reagent volume, and a reagent concentration.

39. The method of claim 30, wherein the data of reagents comprises data of a sequence of a nucleic acid molecule, a volume of the nucleic acid molecule, and a concentration of the nucleic acid molecule.

40. The method of claim 39, wherein the nucleic acid molecule is an oligonucleotide.

41. The method of claim 30, wherein the data of reaction chamber conditions comprises data of a target temperature, a temperature ramp rate, and a time duration at the target temperature.

42. The method of claim 41, wherein the data of reaction chamber conditions comprises specified reaction chamber conditions provided before a run.

43. The method of claim 41, wherein the data of reaction chamber conditions comprises measured reaction chamber conditions provided after a run.

44. The method of claim 30, wherein the polynucleotide quality score provides a level of fidelity of a synthesized polynucleotide sequence compared to a targeted polynucleotide sequence.

45. The method of claim 30, wherein training comprises updating a map of connections of the data of the reagents and the data of the reaction chamber conditions based on the data for the oligonucleotides to improve the polynucleotide quality score.

46. The method of claim 30, wherein the report is provided in a format compatible with a thermocycling device for polynucleotide synthesis.

47. The method of claim 46, wherein the report provides assignments of a reaction mixture and a reaction chamber condition to a reaction chamber of the thermocycling device.

48. The method of claim 46, wherein the method further comprises (e) providing the data of reaction chamber conditions to the thermocycling device; (f) controlling the reaction chamber conditions of an individual reaction chamber of the thermocycling device after the individual reaction chamber is filled with a corresponding reaction mixture based on the report.

49. The method of claim 47, wherein the device comprises a plurality of individual reaction chambers, wherein the reaction chamber conditions of the individual reaction chamber is capable of being controlled independently from another individual reaction chamber in the device.

50. The method of claim 49, wherein the device comprises 96 individual reaction chambers.

51. The method of claim 30, wherein the training takes place in an offline mode, wherein the computing system is not connected to a network.

52. The method of claim 30, wherein the training takes place in an online mode, wherein the computing system is connected to a network.

53. The method of claim 30, further comprising communicating the report to a device for polynucleotide synthesis and providing the reaction chamber conditions to the device.

54. The method of claim 30, wherein the computing system is within a remote server or an external database remote from a user.

55. The method of claim 30, wherein the computing system is within a server or a database local to a user.

56. The method of claim 30, wherein the data are obtained by optical character recognition, voice recognition, touchscreen display input, barcode scanning, or user-initiated data input.

57. The method of claim 49, comprising controlling reaction chamber conditions of a first subset of individual reaction chambers, wherein the first subset is less than the plurality of the reaction chambers, and generating a first result.

58. The method of claim 57, comprising controlling reaction chamber conditions of a second subset of individual reaction chambers based on a second recommendation, wherein the second recommendation is generated using the first result, and generating a second result.

59. The method of claim 58, wherein the second result has a higher polynucleotide quality score than the first result.

60. The method of claim 49, wherein the device comprises a module for measuring the reaction chamber conditions of an individual reaction chamber during a run.

61. The method of claim 60, the module measures measuring the reaction chamber conditions of an individual reaction chamber in real-time.

62. The method of claim 60, the device adjusts the reaction chamber conditions of an individual reaction chamber during the run based on the measured reaction chamber conditions.

63. The method of claim 60, wherein the device adjusts the recommendation based on the measured reaction chamber conditions.

64. The method of claim 63, comprising measuring the deviation of the measured reaction chamber conditions from the specified reaction chamber conditions; correlating the comparison to the polynucleotide quality score; and adjusting the recommendation based on the correlation.

65. The method of claim 60, wherein the device provides a report of the measured reaction chamber conditions.

66. A system for training an artificial intelligence for a polynucleotide synthesis comprising:
(e) at least one processor; and
(f) a memory storing instructions that, when executed by the at least one processor, cause the system to perform:
(i) obtaining data of a sequence of a target polynucleotide, data of oligonucleotides, data of reagents, data of reaction chamber conditions, and a polynucleotide quality score;
(ii) applying, by a computing system, the data to an artificial intelligence;
(iii) training, by the computing system, the artificial intelligence, wherein training comprises:
(1) assigning a connection between one of the data of reagents and one of the data of reaction chamber conditions;
(2) assigning a weight to the connection;
(3) generating a reward signal from the polynucleotide quality score; and
(4) updating the weight based on the reward signal.

67. A system for generating a recommendation of a design of experiment for a polynucleotide synthesis comprising:
(g) at least one processor; and
(h) a memory storing instructions that, when executed by the at least one processor, cause the system to perform:
(i) obtaining data of a sequence of a target polynucleotide and data of oligonucleotides;
(ii) applying, by a computing system, the data to a trained artificial intelligence, wherein training of the artificial intelligence comprises:
(1) assigning a connection between one of data of reagents and one of data of reaction chamber conditions or between one of data of reagents and another of data of reagents;
(2) assigning a weight to the connection;
(3) generating a reward signal from a polynucleotide quality score; and
(4) updating the weight based on the reward signal;
(iii) determining, by the computing system, a recommendation of a design of an experiment by the artificial intelligence, wherein determining comprises:
(1) determining reagents and reaction chamber conditions include in the recommendation based on the data of reagents and the data of reaction chamber conditions in the artificial intelligence; and
(2) determining a sequence of the connections of the data of reagents and the data of reaction chamber conditions that provides a polynucleotide quality score above a threshold score as the recommendation; and
(iv) generating, by the computing system, a report of the recommendation, wherein the recommendation comprises the data of reagents and the data of reaction chamber conditions from the sequence.

68. The system of claim 66, wherein the artificial intelligence comprises machine learning.

69. The system of claim 66, wherein the machine learning comprises reinforcement learning.

70. The system of claim 66, wherein further comprising applying, by the computing system, a dimensionality reduction prior to step (b)(ii).

71. The system of claim 66, wherein the polynucleotide quality score provides a level of fidelity of a synthesized polynucleotide sequence compared to a targeted polynucleotide sequence.

72. The system of claim 66, wherein training comprises updating a map of connections of the data of the reagents and the data of the reaction chamber conditions based on the data for the oligonucleotides to improve the polynucleotide quality score.

73. The system of claim 66, wherein the report is provided in a format compatible with a thermocycling device for polynucleotide synthesis.

74. The method of claim 73, wherein the report provides assignments of a reaction mixture and a reaction chamber condition to a reaction chamber of the thermocycling device.

75. The method of claim 74, wherein the method further comprises (e) providing the data of reaction chamber conditions to the thermocycling device; (f) controlling the reaction chamber conditions of an individual reaction chamber of the thermocycling device after the individual reaction chamber is filled with a corresponding reaction mixture based on the report.

76. The method of claim 75, wherein the device comprises a plurality of individual reaction chambers, wherein the reaction chamber conditions of the individual reaction chamber is capable of being controlled independently from another individual reaction chamber in the device.

77. The method of claim 76, wherein the device comprises 96 individual reaction chambers.

78. A method for polynucleotide synthesis, comprising:
(a) uploading data of a polynucleotide of interest to a computer-based application comprising an artificial intelligence using a computer;
(b) receiving a recommendation of a design of experiment, comprising
(i) determining, by artificial intelligence from the data of the polynucleotide of interest, reagents, reagent conditions, and reaction chamber conditions for polynucleotide synthesis to generate a polynucleotide with a high fidelity; and
(ii) providing a recommendation of reagents, reagent conditions, and reaction chamber conditions for polynucleotide synthesis of the polynucleotide of interest;
(c) selecting reagent conditions and reaction chamber conditions from the recommendation to use in polynucleotide synthesis;
(d) optionally selecting reagent conditions and reaction chamber conditions not provided in the recommendation to use in polynucleotide synthesis, wherein selecting comprises choosing a reagent of interest, conditions for the reagent of interest, and reaction chamber conditions;
(e) preparing reaction mixtures from the selected reagent conditions;
(f) loading the reaction mixtures to the corresponding reaction chambers based on the recommendation;
(g) starting the thermocycling device to perform synthesis of polynucleotide of interest.

79. The method of claim 78, wherein the method further comprises assessing the quality of the synthesized polynucleotide products and uploading the quality to the computer-based application.

80. The method of claim 78, wherein the data of reagents comprises data of a reagent identification, a reagent volume, and a reagent concentration.

81. The method of claim 78, wherein the data of reagents comprises data of a sequence of a nucleic acid molecule, a volume of the nucleic acid molecule, and a concentration of the nucleic acid molecule.

82. The method of claim 81, wherein the nucleic acid molecule is an oligonucleotide.

83. The method of claim 78, wherein the data of reaction chamber conditions comprises data of a target temperature, a temperature ramp rate, and a time duration at the target temperature.
